(19) United States
(12) Patent Application Publication
(10) Pub. No.: US 2010/0004248 A1
(43) Pub. Date: Jan. 7, 2010

(54) Use of a Nitric Oxide Synthase Modulator for the Treatment of Cardiac Indications

(76) Inventors: David Kass, Columbia, MD (US); Eiki Takimoto, Baltimore, MD (US); Hunter Champion, Baltimore, MD (US); Moens An, Baltimore, MD (US)

Correspondence Address: EDWARDS ANGELL PALMER & DODGE LLP, P.O. BOX 55874, BOSTON, MA, 02205, US

(21) Appl. No.: 12/084,165

(22) PCT Filed: Oct. 23, 2006
(86) PCT No.: PCT/US2006/041444

§ 371 Date: Apr. 15, 2009

Related U.S. Application Data
(60) Provisional application No. 60/729,864, filed on Oct. 24, 2005.

(51) Int. Cl. A61K 31/4985; A61P 9/00; A61P 9/04; A61P 9/12; A61P 9/10

(52) U.S. Cl. 514/249

(57) ABSTRACT

The invention features compositions and methods for modulating NOS that are useful for the prevention and treatment of cardiac diseases and disorders, including cardiac hypertrophy and cardiac dilation. In particular, the invention provides compositions comprising tetrahydrobiopterin (BH4), alone or in combination with one or more additional compounds.

### CROSS-REFERENCE TO RELATED APPLICATION

This application claims the benefit of the U.S. Provisional Application No. 60/729,864, the entire contents of which are incorporated herein by reference.

### STATEMENT OF RIGHTS TO INVENTIONS MADE UNDER FEDERALLY SPONSORED RESEARCH

This work was supported by a National Institute of Health Grants PO1-HL59408, HL-47511, and AG18324. The government may have certain rights in the invention.

### BACKGROUND OF THE INVENTION

Chronic cardiac ventricular pressure overload stimulates hypertrophy that can progress to heart failure. A major feature of this transition is pathologic remodeling with chamber dilation and pump dysfunction, and evidence increasingly supports an important role of reactive oxygen species to this process. Reactive oxygen species generation is linked to hypertrophy stimulators such as G_{αq}/G₁₁-coupled agonists (e.g. phenylephrine, angiotensin), signaling kinases and phosphatases, and mechano-transduction. Reactive oxygen species themselves stimulate hypertrophy-associated kinases, induce fetal gene re-expression, and contribute to chamber remodeling by activating matrix metalloproteinases.

Reactive oxygen species can be generated by mitochondrial electron transport leakage, NADPH oxidases, xanthine oxidase, and nitric oxide synthase (NOS). Among these, NOS is of interest since nitric oxide (NO) and its downstream target protein kinase G are generally considered to blunt hypertrophy. However, NOS can be converted to a reactive oxygen species generator as demonstrated in vascular endothelium exposed to increased oxidant or hemodynamic stress. When exposed to oxidant stress, including peroxinitrite (ONOO⁻), or deprived of its reducing cofactor tetrahydrobiopterin (BH4) or substrate L-arginine, NOS3 uncouples to the monomeric form that generates O₂⁻ rather than NO. Uncoupled NOS3 is thought to be a prominent source of endothelial reactive oxygen species in hypertension, neurohormonal stimulation and hyperglycemia, and from ONOO⁻. Hypertension and neurohormonal stress contribute to alterations in multiple cellular signaling and transcription pathways that induce muscle cell growth, worsened function of the heart muscle, hypertrophic remodeling and cardiac dilation. Existing therapies cannot adequately prevent these pathological changes.

Enlargement of the heart is a chronic and progressive condition that ultimately results in heart failure. Heart failure affects over 5 million Americans, with more than 500,000 new diagnoses annually in the United States alone, and remains the leading cause of death. Nearly half of these patients have hypertension and cardiac hypertrophy with apparent preservation of contraction of the heart, a syndrome for which there are currently no specifically tested and approved treatments. Improved therapeutic compositions and methods for the treatment of cardiac conditions, such as cardiac hypertrophy, are urgently required.

### SUMMARY OF THE INVENTION

As described below, the present invention features the use of tetrahydrobiopterin and related compounds, alone or in combination with other therapeutic agents, for the prevention or treatment of cardiac conditions.

In one aspect, the invention features a method of treating a cardiac disease or disorder in a subject (e.g., a human or veterinary patient). The method involves administering to the subject a compound that modulates NOS activity, where the method treats a cardiac disease or disorder.

In another aspect, the invention features a method of enhancing cardiac function in a subject having a cardiac condition selected from the group consisting of cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure. The method involves administering to the subject an effective amount of tetrahydrobiopterin, where the administration of a compound comprising an effective amount of an NOS modulator enhances cardiac function.

In yet another aspect, the invention features a method of treating a cardiac disease or disorder in a subject, the method comprising administering to the subject a compound comprising an effective amount of tetrahydrobiopterin, where the compound modulates NOS3 activity.

In yet another aspect, the invention features a method of treating cardiac hypertrophy in a subject in need thereof. The method involves administering to the subject an effective amount of tetrahydrobiopterin, where the administration of the tetrahydrobiopterin treats cardiac hypertrophy. In one embodiment, the method reduces or reverses cardiac hypertrophy.

In yet another aspect, the invention features method of treating cardiac dilation in a subject in need thereof, the method comprising administering to the subject an effective amount of tetrahydrobiopterin, where the administration of the tetrahydrobiopterin treats cardiac dilation. In one embodiment, the method reduces or reverses cardiac dilation.

In yet another aspect, the invention features a method of treating or preventing a cardiac disease or disorder in a subject. The method involves administering to the subject an effective amount of a combination of tetrahydrobiopterin and at least one compound selected from the group consisting of a PDE5 inhibitor, an anti-oxidant, folate, YC-1, BAY 58-2667, BAY 41-2272, or BAY-41-8543, where the administration of the combination treats or prevents a cardiac disease or disorder. In one embodiment, the cardiac disease or disorder is selected from the group consisting of cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure. In another embodiment, at least two, three, four, five, or six compounds are administered. In yet another embodiment, tetrahydrobiopterin and a PDE5 inhibitor are administered in amounts sufficient to prevent or treat cardiac hypertrophy or cardiac dilation.

In yet another aspect, the invention features a pharmaceutical composition comprising an effective amount of tetrahydrobiopterin in a pharmaceutically acceptable excipient, where the pharmaceutical pack is labeled for use in the treatment or prevention of a cardiac disease or disorder.

In yet another aspect, the invention features a pharmaceutical composition comprising an effective amount of tetrahydrobiopterin and at least one compound selected from the group consisting of at least one compound selected from the group consisting of a PDE5 inhibitor, an anti-oxidant, folate, YC-1, BAY 58-2667, BAY 41-2272, or BAY-41-8543, in a pharmaceutically acceptable excipient, where the pharmaceutical pack is labeled for use in the treatment or prevention of a condition selected from the group consisting of a cardiac disease or disorder. In one embodiment, the cardiac disease or disorder is cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling or cardiac failure. In one embodiment, the tetrahydrobiopterin or the combination is provided in a sustained release formulation. In other embodiments, the composition further includes written instructions for administering the composition to a subject for the treatment or prevention of a cardiac disease or disorder.

In other aspects, the invention features a kit for the treatment of a cardiac disease or disorder comprising tetrahydrobiopterin and any one or more of PDE5 inhibitor, an anti-oxidant, folate, YC-1, BAY 58-2667, BAY 41-2272, or BAY-41-8543 and directions for their use in the treatment or prevention of a cardiac disease or disorder.

In another aspect, the invention features a method of preventing a cardiac disease or disorder in a subject, the method comprising administering to the subject a compound that modulates NOS activity, where the method treats a cardiac disease or disorder.

In yet another aspect, the invention features a method of preventing a cardiac disease or disorder in a subject, the method comprising administering to the subject a compound comprising an effective amount of tetrahydrobiopterin, where the compound modulates NOS3 activity.

In yet another aspect, the invention features a method of treating or preventing a cardiac disease or disorder in a subject in need thereof, the method involving administering to the subject an effective amount of folic acid or a metabolite thereof (e.g., 5-methyltetrahydrofolate), wherein the administration of the folic acid or a metabolite thereof treats or prevents the cardiac disease or disorder.

In yet another aspect, the invention features a method of enhancing cardiac function in a subject having a cardiac condition (e.g., cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure), the method involving administering to the subject an effective amount of folic acid or a metabolite thereof, wherein the administration of the compound enhances cardiac function. In one embodiment, the method further involves administering to the subject sildenafil or tetrahydrobiopterin.

In another aspect, the invention features a pharmaceutical composition comprising an effective amount of folic acid or a metabolite thereof in a pharmaceutically acceptable excipient, where the pharmaceutical composition is labeled for use in the treatment or prevention of a condition selected from the group consisting of a cardiac disease or disorder. In one embodiment, the composition further contains at least one of a PDE5 inhibitor, an anti-oxidant, or tetrahydrobiopterin, in a pharmaceutically acceptable excipient,

In various embodiments of any of the above aspects, the cardiac disease or disorder is any one or more of cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure. In other embodiments of any of the above aspects, the compound modulates NOS3 activity (e.g., reduces NOS3-dependent generation of reactive oxygen species, increases NO production, or does not reduce NO production). In still other embodiments of any of the above aspects, the NOS modulator is tetrahydrobiopterin (BH4), folic acid (folate), 5-HTMF (5-Methyl-tetrahydrofolate), reducing agents (e.g. superoxide dismutase, TEMPOL, n-acetyl cysteine), or anti-oxidants (e.g. resveratrol, Vitamin C, cyaniding). In yet other embodiments of any of the above aspects, the compound is tetrahydrobiopterin. In still other embodiments of any of the above aspects, the method reduces or reverses cardiac chamber remodeling, cardiac dilation, cardiac muscle cell remodeling (e.g., reduces myocyte size), myocyte hypertrophy, molecular remodeling (e.g., the method reduces re-expression of a fetal gene, such as B-natriuretic peptide or α-skeletal actin), myocardial fibrosis, or oxidative stress.

In other embodiments of any of the above aspects, the method reduces nitric oxide synthase uncoupling, reduces production of reactive oxygen species, or reduces cardiac gelatinase activity, oxidative stress-linked stimulation of protein kinase, sarcomere protein oxidation, or other adverse consequences of oxidative stress in the cardiac myocyte. In yet other embodiments of the above aspects, the method enhances cGMP-dependent signaling. In still other embodiments of any of the above aspects, the cardiac chamber, cellular or molecular remodeling is induced by a stimulus (e.g., pressure-overload, neurohormonal stress, myocardial infarction, volume-overload). In yet other embodiments of the above aspects, the method involves assessing cardiac function, for example, by measuring relaxation rate independent of load, cardiac contractility independent of load; cardiac ejection volume independent of load, end-systolic volume independent of load. In still other embodiments, cardiac function is determined using any one or more of the following assays: Doppler echocardiography, 2-dimensional echo-Doppler, Pulse-wave Doppler, continuous wave Doppler, oscillometric arm cuff, cardiac catheterization, magnetic resonance imaging, positron emission tomography, chest X-ray, ejection fraction test, electrocardiogram, nuclear scanning, invasive cardiac pressures, invasive and non-invasively measured cardiac pressure-volume loops (conductance catheter). In still other embodiments of the above aspects, the method further includes the step of administering to the subject a PDE5 inhibitor in combination with a compound that reduces NOS3-dependent production of reactive oxygen species, anti-oxidant, folate, a compound that activates a soluble guanylate cyclase (e.g., YC-1, BAY 58-2667, BAY 41-2272, or BAY-41-8543. In various embodiments of any of the above aspects, combinations of the invention are administered concurrently, or one compound of the invention is prior to the other. For example, in some embodiments, tetrahydrobiopterin is administered at least about 3, 5, or 7 days prior to the PDE5 inhibitor, 1, 2, 3 or 5 weeks prior to the PDE5 inhibitor, or at least about 1 or 2 months prior to the PDE5 inhibitor. In other embodiments, a PDE5 inhibitor is administered prior to the administration of tetrahydrobiopterin, (e.g., at least about 3, 5, or 7 days prior to BH4, 1, 2, 3 or 5 weeks prior to BH4, or at least about 1 or 2 months prior to BH4.

The invention provides compositions and methods for the treatment of cardiac diseases or disorders featuring tetrahydrobiopterin. Other features and advantages of the invention will be apparent from the detailed description, and from the claims.

### DEFINITIONS

By “NOS modulator” is meant a compound or combination of compounds that alters NOS activity. Exemplary NOS modulators include, but are not limited to, tetrahydrobiopterin (BH4), folic acid (folate), 5-HTMF (5-Methyl-tetrahydrofolate), reducing agents (e.g. superoxide dismutase, TEMPOL, n-acetyl cysteine), anti-oxidants (e.g. resveratrol, Vitamin C, cyaniding). In one embodiment, an NOS modulator reduces NOS uncoupling or increases NO production. Exemplary nitric oxide synthases include NOS isoform 1, 2, or 3.

By “NOS activity” is meant any NOS enzymatic function. Exemplary functions include the generation of NO or the generation of reactive oxygen species (ROS).

By “NOS uncoupling” is meant the transition of nitric oxide synthase enzyme so that its primary synthetic mode (conversion of L-arginine to L-citrulline with the production of nitric oxide) is altered to increase its generation of reactive oxygen species. This can be associated with its transition from a homodimeric to a monomeric form.

By “anti-oxidant” is meant a compound that reduces oxidation, that reduces free radical production, or that inhibits a reaction associated with a free radical. Exemplary anti-oxidants include vitamin C, superoxide dismutase, n-acetyl cysteine, oxypurinol, reduced glutathione (GSH), vitamin E, and TEMPOL.

By “activates” is meant increases the expression or activity of a polypeptide or nucleic acid molecule.

By “cardiac hypertrophy” is meant any undesirable cardiac muscle cell growth, increase in cardiac chamber mass relative to body size, or increase in cardiac chamber wall thickness at normal or increased chamber volume.

By “cardiac condition” is meant any cardiac disease or disorder. Exemplary cardiac diseases include, but are not limited to, cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure.

By “cardiac chamber remodeling” is meant an undesirable morphological alteration in a cardiac tissue in response to a pathophysiologic stimulus (e.g., hypertension, myocardial infarction, neurohormonal stress, volume over-load). Examples of cardiac chamber remodeling include increase in cardiac hypertrophy and a sustained increase in cardiac chamber dimensions—i.e. pathological cardiac dilatation—associated with an increase in the unstressed cardiac volume.

By “cellular remodeling” is meant an undesirable alteration in a cardiac cell in response to a pathophysiologic stimulus. Changes in cellular remodeling include, but are not limited to, changes in any one or more of the following: myocyte hypertrophy, myocyte elongation and thinning (e.g. morphologic changes typical of cardiac failure), interstitial fibrosis, changes in excitation-contraction coupling including altered calcium handling (e.g., cyclic changes in intracellular calcium with myocyte stimulation, uptake and release of calcium from internal cellular stores, such as the sarcoplasmic reticulum, interaction of calcium with a contractile protein or regulatory protein), activating current (e.g., sodium), and repoloarizing current (e.g., potassium).

By “molecular remodeling” is meant an alteration in the transcription and/or expression of a gene or an alteration in the biological activity of the synthesized protein (post-translational modification) in cardiac tissue in response to a pathophysiologic stimuli.

By “enhancing cardiac function” is meant producing a beneficial alteration in the pumping performance and capacity of the heart.

By “maladaptive cardiac alteration” is meant an undesirable change in the heart, or in a cell thereof, in response to a pathophysiologic stimulus.

By “modulate” is meant a positive or negative alteration.

By “PDE5 inhibitor” is meant a compound that inhibits cGMP hydrolysis by phosphodiesterase-5. PDE5 inhibitors preferably reduce PDE5 enzymatic activity by at least 5% (e.g., 10%, 15%, 20%, 30%, 50%, 60%, 75%, 85%, 90% or 95%). Methods for assaying the activity of a PDE5 inhibitor are known in the art and are described herein (e.g., at Example 4).

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “treat” is meant decrease, suppress, attenuate, diminish, arrest, or stabilize the development or progression of a disease.

By “disease” is meant any condition or disorder that damages or interferes with the normal function of a cell, tissue, or organ.

By “modulation” is meant any alteration (e.g., increase or decrease) in a biological function or activity.

By “reduce” or “increase” is meant alter negatively or positively, respectively, by at least 5%. An alteration may be by 5%, 10%, 25%, 30%, 50%, 75%, or even by 100%.

By “reduces cardiac hypertrophy” is meant produces at least a 5% decrease in a morphological, cellular, or molecular remodeling.

By “reverses cardiac hypertrophy” is meant produces a desirable alteration in a morphological, cellular, or molecular cardiac phenotype, wherein the altered phenotype is substantially that characterizing normal cardiac tissue.

By “subject” is meant a mammal, such as a human patient or an animal (e.g., a rodent, bovine, equine, porcine, ovine, canine, feline, or other domestic mammal).

An “effective amount” is an amount sufficient to effect a beneficial or desired clinical result.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### BRIEF DESCRIPTION OF THE DRAWINGS

FIGS. 1A-1D show that genetic lack of NOS3 ameliorates cardiac hypertrophy and dilatory remodeling in response to trans-aortic constriction (TAC) induced pressure-overload. FIG. 1A shows six cross-sections through 10% formalin fixed hearts of wild-type and mice lacking NOS3 (NOS3^{-/-}) hearts subjected to transverse aortic constriction. The three wild-type hearts at the top of the panel show cardiac hypertrophy developing marked dilatory remodeling over time while the three NOS3^{-/-}-TAC hearts at the bottom of the panel show concentric cardiac hypertrophy at 3 weeks with no further progression at 9 weeks. Scale bar represents 10 mm. FIG. 1B is a graph showing mean data for heart weight/tibia length (HW/TL) ratio (n≧6 for each group). FIG. 1C is a series of six micrographs showing a histological analysis of wild-type and NOS3^{-/-}-TAC hearts. Periodic acid-Schiff (PAS) methenamine staining reveals increased interstitial fibrosis (black, upper right) and myocyte size in wild-type-TAC. NOS3^{-/-}-TAC hearts reveal minimal fibrosis and blunted increase in myocyte size. Scale bar represents 100 μm. FIG. 1D is a graph showing a summary quantification of cardiomyocyte diameter (n=4-5 each genotype, 6-10 regions per heart, n=50-60 cells/heart for size estimates); p-values are for interaction of TAC and genotype based on 2-way ANOVA. Throughout the figures, “WT Sham 3W” and “WT Sham 9W” denotes a sham operated animal at 3 weeks and 9 weeks, respectively; “WT TAC 3W and WT TAC 9W” denotes a wild-type TAC animal at 3 weeks and 9 weeks post-surgery; “NOS3^{-/-} Sham 3W” “NOS3^{-/-} Sham 9W” denotes a sham operated animal lacking NOS3^{-/-} at 3 weeks and 9 weeks; and “NOS3−/− TAC 3W” “NOS3^{-/-}-TAC 9W” denotes a NOS3^{-/-}-TAC animal at 3 weeks and 9 weeks post-surgery.

FIGS. 2A and 2B show in vivo hemodynamics in wild-type and NOS3^{-/-} hearts subjected to TAC. FIG. 2A representative pressure-volume (PV) loops pressure-volume (PV) loops and end-systolic and end-diastolic relations (dashed lines). In wild-type-TAC, the PV relations shifted rightward modestly at 3 weeks and markedly at 9 weeks, whereas the opposite occurred in NOS3^{-/-}-TAC. Left ventricular systolic pressures were similarly increased (at 3 weeks). The upper left relation (end-systolic PV relation) was steeper in NOS3^{-/-}-TAC than in wild-type-TAC. Comprehensive analysis is provided in Table 1. FIG. 2B (top panel) shows M-mode echocardiography in conscious animals demonstrating dilated hypertrophy with decreased fractional shortening in wild-type-TAC, but concentric hypertrophy with preserved shortening in NOS3^{-/-}-TAC. FIG. 2B (bottom panel) is a graph showing summary data from echocardiography (n≧5 for each group). Wall thickness increases similarly by TAC at 3 weeks between genotypes, and at longer time period (9 weeks), decreases slightly in wild-type and remains unchanged in NOS3^{-/-}. Chamber end-diastolic (EDD) and end-systolic (ESD) dimensions and fractional shortening (% FS) markedly differed between the genotype. P-values are for interaction of TAC and genotype based on 2-way ANOVA.

FIGS. 3A and 3B provide an analysis of fetal gene expression in left ventricles. FIG. 3A provides a dot blot analysis of fetal gene expression in left ventricles. FIG. 3B is a series of 6 graphs that provide summary quantification, with results normalized by GAPDH (n=34 for each group). P value in each plot reflects the interaction of TAC response and genotype, based on 2-way ANOVA. Throughout the figures “ANP” denotes type-A (atrial) natriuretic peptide; “BNP” denotes type-B (brain) natriuretic peptide; “β-MHC” denotes β-myosin heavy chain, α-SA denotes α-skeletal actin; “PLB” denotes phospholamban and “SERCA2α” denotes sarcoplasmic reticulum Ca²⁺ ATPase.

FIGS. 4A-4D show reactive oxygen species (ROS) levels in wild-type and NOS3^{-/-} hearts subjected to TAC. FIG. 4A is a graph showing the results of a luminol chemiluminescence assay for superoxide in myocardial tissue extracts. TAC stimulated O₂⁻ formation in wild-type hearts, but far less in NOS3^{-/-} hearts. P-values are for interaction of TAC and genotype based on 2-way ANOVA. FIG. 4B is a series of four micrographs showing intracellular ROS generation as estimated by red dihydroethidium (DHE) staining. FIG. 4C is a series of four micrographs showing green 2′,7′-dichlorofluorescein (DCF) staining in frozen sections imaged by confocal fluorescent microscopy. Both signals were increased in wild-type-TAC, and strongly attenuated in NOS3^{-/-}-TAC. FIG. 4D is a series of four micrographs showing nitrotyrosine (NT) measured by immunofluorescent staining and quantified by ELISA assay. Both methods revealed a marked increase in NT in WT-TAC, but low levels in NOS3^{-/-}-TAC, as in controls for both genotypes. *p<0.05 vs other groups. Scale bars represent 50 μm.

FIGS. 5A-5C show reduced/oxidized glutathione (GSH/GSSH) levels, matrix metalloproteinase (MMPs) and Akt activation. FIG. 5A is a series of three graphs showing a quantitation of high-performance liquid chromatography determination of reduced/oxidized glutathione (GSH/GSSH) ratio, xanthine, and reduced NADP (NADPH). GSH/GSSH markedly declined with TAC in wild-type hearts, but not NOS3^{-/-} hearts. Xanthine increased in both, but somewhat more in wild-type, while NADPH declined similarly in both genotypes. *p<0.05 vs sham hearts. FIG. 5B shows gelatin zymography of myocardium in controls and following three weeks TAC (left panel), and a quantification of the results (graph, right panel). Positive control (+C) bands for activated MMP-2 and MMP-9 are shown. Basal gel lysis was minimal, but markedly increased in wild-type-TAC. This was not observed in NOS3^{-/-} heart either at baseline, or with TAC. *p<0.05 vs other groups. In FIG. 5B “+C” denotes a positive control for MMP-2 and MMP-9. FIG. 5C (left panel) is a Western blot showing the response of total Akt (t-Akt) and phosphorylated Akt (p-Akt) to TAC in wild-type and NOS3^{-/-} hearts. FIG. 5C (right panel) is a graph showing the quantification results as a ratio of phospho- to total Akt (n=3 for each group). TAC induced marked increase in p/t Akt levels in wild-type heart. In contrast, there was no change in NOS3^{-/-} hearts. *p<0.01 vs wild-type sham 3W.

FIGS. 6A-6D show NOS3 uncoupling in wild-type (WT)-TAC hearts. FIGS. 6A and 6B are Western blots in a non-reducing gel showing that in wild-type sham heart, NOS3 appeared as both a dimer (NOS3-d) and a monomer (NOS3-m), with the largest fraction as a dimer. In boiled samples (control), the dimer was replaced by the monomeric form. 3 weeks WT-TAC heart exhibited largely the monomeric form, although total NOS3 expression assessed by Western blot (FIG. 6B) was not altered. FIG. 6C is a graph showing NOS calcium dependent and independent activity based on L-citrulline formation. Ca²⁺-dependent activity declined in WT-TAC (*p<0.05). Low levels were also seen in NOS3^{-/-} mutants reflecting NOS1 activity. Ca²⁺ independent NOS2 activity was little changed. FIG. 6D is a graph showing the impact of pharmacological NOS3 inhibition on luminol chemiluminescence assay. Co-incubation with 1 mM LNAME inhibited 50% of luminol chemiluminescence in 3 weeks and 9 weeks WT-TAC heart lysates, while it inhibited <15% at baseline, supporting an increased role of NOS to O₂⁻ generation with TAC. *p<0.05 vs sham 3W.

FIGS. 7A-7H show that tetrahydrobiopterin (BH4), but not tetrahyrdoneopterin (H₄N), prevents NOS3 uncoupling, ROS generation and cardiac remodeling induced by 3 weeks TAC. FIG. 7A (top panel) shows cross sections of 10% formalin fixed hearts; FIG. 7A (middle panel) is a series of two micrographs (PAS methenamine) showing concentric hypertrophy with BH4 co-treatment versus dilative hypertrophy with H₄N accompanied by increased interstitial fibrosis. Scale bars represent 10 mm for upper panel and 100 μm for lower panel. FIG. 7B shows representative M-mode echocardiography and. FIG. 7C shows representative PV loops from wild-type animals treated with BH4 following TAC. These studies reveal corresponding functional improvement in BH4 but not H₄N-treated hearts. FIG. 7D is a Western blot showing that NOS3 dimer (NOS3-d) was preserved in BH4-treated but not in H₄N-treated hearts. FIG. 7E is a graph showing that NOS Ca²⁺-dependent activity was restored by BH4 but not H₄N treatment. *p<0.05 vs sham. FIG. 7F (upper panel) is a graph that quantitates luminol chemiluminescence. This study detects a decline in O₂⁻ generation in WT-TAC hearts treated with BH4, but minimal effect with H₄N treatment. *p<0.05 vs sham. FIG. 7F (lower panel) is a graph that shows the percent of luminol signal blunted by co-incubation with L-NAME, confirming reduced NOS-derived O₂⁻ in BH4 treated hearts. *p<0.05 vs BH4. Bar graph labeling is the same as indicated in (e). FIG. 7G is a series of four confocal images of DHE (red) and DCF (green) stained myocardium from WT-TAC hearts treated with either BH4 or H₄N. Scale bar represents 50 μm. FIG. 7H shows gelatin zymography for hearts with BH4 or H₄N treatment and quantification results. The increased gel lysis in WT-TAC was reduced by BH4, but not H₄N therapy. *p<0.05 vs sham. Bar graph labeling is the same as indicated in (e).

FIGS. 8-23 describe studies showing that BH4 treatment initiated after establishment of substantial cardiac hypertrophy, dilation, and remodeling can be reversed. FIG. 8 is a photograph of gross heart specimens that illustrate that delayed BH4 treatment of chronic pressure-overload reverses chamber dilation and hypertrophy. Two examples of gross heart specimens are shown for each condition, including baseline (control), after 9-weeks of trans-aortic constriction (9 weeks-TAC), and after 9 weeks of TAC, with BH4 treatment initiated at week 5 (BH4 rev 9 weeks TAC).

FIG. 9 shows cross sectional histology (3 top panels) and microscopic histology (3 bottom panels) of a control heart, a heart after 9 weeks of TAC (9 weeks), and 9-weeks of TAC with BH4 treatment started at week 5 (9 weeks BH4). The sections again show marked reversal of hypertrophy and reduced chamber size. Myocyte size is markedly reduced (lower panels) compared to the untreated heart.

FIG. 10 is a graph that summarizes the effects of BH4 treatment on myocyte cross section dimension in sham control, hearts exposed to 4-weeks of TAC, hearts exposed to 9 weeks of TAC< and hearts exposed to 9 weeks of TAC with BH4 administered from weeks 5-9 (BH4 rev 9 weeks TAC). There is a significant reduction in myocyte size with BH4 treatment, with cell size even less than it was at the onset of this therapy (i.e. smaller than after 4 weeks of TAC). Thus, BH4 treatment reverses myocyte hypertrophy.

FIG. 11 shows echocardiograms (m-mode) for a control mouse heart, a heart with a sham surgical procedure (sham 9 weeks), a heart after 4 weeks of TAC (TAC 4 weeks), and a heart after 9 weeks of TAC (TAC 9 weeks), and a heart after 9 weeks TAC with BH4 treatment started after week 5 (TAC 92k+BH4 rev). BH4 treatment markedly reduced chamber size improved myocardial function.

FIG. 12 data for left ventricular ejection fraction (EF) and fractional shortening (FS) in control mice, mice exposed to sham operation, and mice exposed to TAC at 4 weeks, 9 weeks, and 9 weeks with BH4 treatment started at week 5. EF and FS were both enhanced in the BH4 treatment group compared to non-treated 9 weeks TAC hearts, and were even improved compared with hearts after 4 weeks TAC. Thus BH4 treatment reverses chamber dysfunction due to chronic pressure-overload (TAC).

FIG. 13 shows echocardiographic dimension and wall thickness measurements for the same protocols described in FIG. 12. BH4 treatment reduced diastolic wall thickening, and both LV end-systolic and end-diastolic dimension compared with control hearts following 9 weeks TAC. The terms used in the figures are defined as follows: LVEDD—left ventricular end-diastolic dimension; LVWTdias-LV wall thickness in diastole); IVSdiast—intraventricular septal thickness in diastole; LVESD—left ventricular end-systolic dimension.

FIG. 14 is a series of three graphs showing echocardiographic calculated LV mass, measured heart weight, and heart weight to body weight ratio for same protocols as in FIG. 12. BH4 treatment reduced LV mass and measured heart weight, as well as reducing the heart weight/body weight (HW/BW) ratio.

FIG. 15 shows re-coupling of nitric oxide synthase (NOS) by BH4 treatment of advanced hypertrophic/dilated hearts induced by 4 weeks TAC. FIG. 15, upper panel, shows a gel electrophoresis of NOS dimer (280 kD) and monomer (140 kD), demonstrating increased monomer following 9 weeks TAC (NOS uncoupling). Uncoupling is reversed and levels of NOS dimer are restored to normal by BH4 treatment. Bar graphs in the lower panel summarize densitometry results from the gel in the upper panels and 3 additional similar gels. The ratio of dimer/monomer for eNOS increased with BH4 treatment. There was no change in total protein (lower right panel).

FIG. 16 shows reduced myocardial fibrosis resulting from BH4 treatment of chronic pressure-overloaded heart. The upper panels show myocardial histology using a fibrosis stain (darker color). While minimal fibrosis is observed in control hearts, increasing levels of interstitial fibrosis are observed following 4 and 9 weeks of TAC. BH4 treatment reduced interstitial fibrosis observed in tissues harvested at 9 weeks when treatment was initiated at 4 weeks TAC and continued for 5 weeks. The lower panel quantitates fibrosis in these tissues and confirms that BH4 treatment reduced fibrosis to essentially normal levels.

FIG. 17 shows that superoxide levels increased as evidenced by dihydroethidium (DHE) staining. The upper panel shows myocardial sections stained with DHE. The lower panel provides a graph that quantitates these results. At 9 weeks TAC, there was a marked increase in oxidative stress (light gray nuclei reflect positive DHE staining for superoxide). This was largely reversed by treatment with BH4 initiated at four weeks and continued for 5 weeks.

FIG. 18 shows that BH4 treatment improves cardiomyocyte function. FIG. 18 includes four exemplary tracings showing sarcomere shortening (top) and calcium transients (bottom) from a myocyte following 9-weeks TAC heart without treatment (No Rx), and one in which BH4 treatment was initiated at week 4 and continued for 5 weeks (+BH4). There is a slight increase in calcium transient amplitude and faster calcium transient decay along with more rapid rise and decay of sarcomere shortening. These tracings show that the kinetics of myocyte contraction and calcium handling are improved by BH4 treatment.

FIG. 19 is a table summarizing myocyte shortening and calcium transient data.

FIG. 20 shows in vivo pressure-volume loops for a control heart, a heart following 9-weeks of TAC, and a heart treated with BH4, where treatment was initiated at week 4 of TAC and continued for 5 weeks. The untreated chronic TAC heart displayed a marked increase in volume and depressed heart function, with the pressure-volume loops and relations shifting markedly to the right (consistent with marked remodeling). In contrast, the heart treated with BH4 has essentially normal heart volumes, and improved systolic function relative to the untreated control. Note that the degree of increased systolic pressure was similar in both treated and untreated hearts following 9 weeks TAC. For useful comparison, the pressure-volume loops at 3 wks TAC (shown in FIG. 2a) should be reviewed. This shows that at the time of BH4 treatment (i.e. after 4 wks of TAC), there is already substantial rightward shift of the pressure-volume data consistent with existing remodeling and dysfunction. Thus, the current data show BH4 reverses this remodeling.

FIG. 21 is a table that summarizes in vivo hemodynamic data in control (n=6), 9 weeks TAC (n=4), and 9 weeks TAC (n=5) with BH4 treatment (where treatment was initiated at week four and continued for 5 weeks). Statistical differences are provided to the right. The terms used in the figures are defined as follows: HR—heart rate; LVP peak—peak left ventricular pressure; LVP end-diastolic—left ventricular end-diastolic pressure; LVV end-systolic—left ventricular end-systolic volume; LVV end-diastolic—left ventricular end-diastolic volume; SV —stroke volume; CO—cardiac output; dP/dtmax—maximal rate of pressure rise; dP/dtmin—maximal rate of pressure decline; Ea—effective arterial elastance (afterload); PWRmax/EDV—maximal ventricular power index; Et Tau-1—time constant of relaxation of chamber stiffening; dP/dt-ip—maximal rate of pressure rise normalized to instantaneous developed pressure; PRSW —preload recruitable stroke work (contractility index); and Ees/heart weight—end-systolic elastance normalized to heart mass (contractility parameter).

FIG. 22 shows graphs of oxidant stress detected by luminol assay and superoxide in particular detected by lucigenin assay. For the luminol assay, data are shown for in control, sham operated, and TAC animals at 4 weeks, 9 weeks TAC animals, and 9 week TAC animals treated with BH4 starting in week 5 and lasing for the remaining 5 weeks. For the lucigenin assay, data are shown for control, and 9 weeks TAC either without, or with the delayed BH4 treatment.

FIG. 23A is a graph showing nitric oxide synthase activity measured to radiolabeled arginine-citrulline conversion assay in myocardial tissue extract from control, 9 weeks TAC, and 9 weeks TAC treated with BH4 during the last 5-week period. BH4 treatment improved the activity of nitric oxide synthase in myocardium exposed to sustained pressure overload. FIG. 23B shows the amount of super-oxide formation due to NOS uncoupling. Data were generated by lucigenin assay, incubating tissue with the NOS inhibitor L-NAME, and then determining what percent of the signal was eliminated by inhibiting NOS. Data are shown for control, 9 week TAC, and 9 week TAC with BH4 treatment during the last 5 week period. 9 week TAC results in a rise in superoxide formation due to NOS, and this was reduced to control levels by delayed BH4 treatment.

FIGS. 24A-D show the effect of folic acid (folate) treatment on preventing the development of ventricular hypertrophy and late cardiac remodeling after 9 weeks of TAC. Data are shown at baseline, and after 3 and 9 weeks of TAC in untreated and treated animals. FIG. 24A compares the effect on actual measured heart weight at terminal study (9 wks) and 24B on calculated heart mass (echocardiography) at both 3 and 9 wks TAC. Cardiac mass (hypertrophy) was reduced by folate. FIG. 24C shows ejection fraction at both 3 and 9 weeks of TAC; FIG. 24D shows left ventricular end-diastolic dimension. Ejection fraction was increased, cardiac dilation prevented by co-treatment with folate during the 9 week TAC period.

FIG. 25 is a graph showing that the capacity of PDE5a inhibition to blunt the beta-adrenergic response in cardiac muscle cells (myocytes) exposed to isoproterenol. Comparison is made between control cells, cells obtained from 3 week TAC hearts (TAC), and cells obtained from 3 week TAC hearts with pre-treatment with the reducing agent (reduced glutathione; TAC+GSH).

FIG. 26 is a graph showing the ability of sustained activation of soluble guanylate cyclase to restore the anti-adrenergic effect of a PDE5a inhibitor (sildenafil, SIL) in intact hearts with chronically suppressed nitric oxide synthase. The bars at the far left show that the isoproterenol response is inhibited by sildenafil (SIL). In hearts exposed to the NOS inhibitor L-NAME for 1 week, sildenafil no longer suppresses ISO stimulated contractility, and acute administration of a soluble guanylate cyclase activator (BAY 41-8543) does not reverse this (middle bars). However, if BAY 41-8543 is administered for a week while maintaining inhibition of NOS, the ISO response can once again be suppressed by SIL (rightward bars). This indicates that enhancers of NOS-related signaling can augment the physiologic regulation of heart function by PDE5a inhibitors.

FIG. 27 includes two graphs showing that chronic BH4 treatment and chronic PDE5a (sildenafil) inhibitor treatment act through different mechanisms in hearts exposed to sustained pressure overload. FIG. 27 (right panel) shows protein kinase G activity (PKG) in control (con) hearts or hearts subjected to 9 weeks of TAC in the presence (tacbh4) or absence (tac9wk) of BH4 treatment.

### DETAILED DESCRIPTION OF THE INVENTION

The invention features compositions and methods for modulating NOS that are useful for the prevention and treatment of cardiac diseases and disorders, including cardiac hypertrophy and cardiac dilation. In particular, the invention provides compositions comprising BH4 or BH4 in combination with one or more compounds that enhance BH4 efficacy, stabilization, salvage, and/or that increase cGMP levels. In other embodiments, BH4 is provided in combination with a PDE5 inhibitor.

This invention is based, in part, on the discovery that NOS3 uncoupling occurred in myocardium exposed to chronic pressure-load, and that this serves as a major source for myocardial reactive oxygen species. Reactive oxygen species are linked to dilative hypertrophy remodeling. In addition, oral supplementation with BH4 was found to prevent NOS3 uncoupling, and markedly blunted reactive oxygen species generation and chamber dilation despite similar levels of chronic chamber loading. Surprisingly, BH4 treatment was also found to reverse cardiac hypertrophy and cardiac dilation. Accordingly, compositions and methods of the invention are particularly useful for the treatment or prevention of cardiac conditions that are characterized by morphological, cellular, or molecular remodeling. Typically, such remodeling occurs in response to hemodynamic stress such as hypertension, valvular disease, neurohormonal stress, cardiac infarction, or volume over-load.

The present invention provides methods of treating cardiac disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a cardiac disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which NOS3 in reactive oxygen species generation may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with NOS uncoupling, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Prophylactic and Therapeutic Applications

Heart disease is typically a chronic and progressive illness that kills more than 2.4 million Americans each year. There are ˜500,000 new cases of heart failure per year, with an estimated 5 million patients in the United States alone having this disease. Early intervention is likely to be most effective in preserving cardiac function. Desirably, methods of the invention are used to prevent as well to reverse the morphological, cellular, and molecular remodeling that is associated with heart disease. In one embodiment, heart disease is prevented by administering an effective amount of an agent that modulates NOS. For example, a compound that reduces NOS3 uncoupling, such as tetrahydrobiopterin (BH4), or a combination of the invention (e.g., BH4 and at least one of an anti-oxidant, folate, a PDE5A inhibitor, and a soluble guanylate cyclase activator) are administered to a subject having or at risk of developing a cardiac condition. To determine a subject's propensity to develop a cardiac condition, the subject's cardiac risk is assessed using any standard method known in the art. The most important indicators of cardiac risk are age, hereditary factors, weight, smoking, blood pressure, diet, exercise history, and diabetes. Other indicators of cardiac risk include the subject's lipid profile, which is typically assayed using a blood test, or any other biomarker associated with heart disease or hypertension, for example C-reactive protein. Other methods for assaying cardiac risk include, but are not limited to, an EKG stress test, thallium stress test, EKG, CT scan, echocardiogram, magnetic resonance imaging study, non-invasive and invasive arteriogram, and cardiac catheterization.

Agents that reduce NOS3 uncoupling, such as tetrahydrobiopterin (BH4) or a combination of the invention (e.g., BH4 and at least one of an anti-oxidant, folate, a PDE5A inhibitor, and a soluble guanylate cyclase activator) are also useful for treating maladapative cardiac alterations that involve chamber, cellular, and molecular remodeling leading to cardiac dysfunction, hypertrophy, and dilation, and by other cardiac indications. Advantageously, the methods of the invention are useful for the reduction of morphological, cellular and molecular remodeling in cardiac tissues that are under stress related to pressure-overload, neurohormonal stress, myocardial infarction, or volume-overload. Accordingly, the methods of the invention are particularly useful in patient's having uncontrolled hypertension or any other chronic condition that places stress on the heart.

### Nitric Oxide and Nitric Oxide Synthase

NO synthesis is catalyzed by the enzyme NO synthase (NOS). Three types of NOS have been identified; type I NOS is found at high concentrations in nervous tissue; type II NOS, or inducible NOS, is induced in response to immunological challenge; and Type III NOS, or endothelial NOS, is activated by Ca²⁺/calmodulin and by phosphorylation by protein kinases. NOS3 is the dominant isoform of nitric oxide synthase present in the endothelium as well as in cardiac myocytes. NO serves as an intercellular messenger that activates soluble guanylate cyclase, thereby increasing levels of cGMP and inducing relaxation of smooth muscle cells. NO biosynthesis involves the conversion of arginine into free NO, a free radical gas, and citrulline in a reaction that is catalyzed by NOS and that requires a tetrahydrobiopterin cofactor and NADPH. Uncoupling of NOS leads to overproduction of free radicals, including superoxide. Free radicals are highly reactive molecules that posses an outer electron orbital with a solitary unpaired electron. While some level of oxidant species is considered normal and important participants in cell signaling, excessive production leads to oxidative stress, a pathological condition that damages cells and tissues when cellular antioxidant defenses are inadequate to completely detoxify the free radicals being generated.

When produced in excessive amounts, NO itself can serve as a ROS, but at physiologic and pharmacologic concentrations, its radical activity is limited. However, NO can combine with superoxide, O(2)(−), to produce a highly oxidizing compound, peroxynitrite (ONOO(−)). Peroxynitrite reacts with protein tyrosine residues to produce nitrotyrosine. Nitrotyrosine disrupts cellular metabolism by inactivating a number of important cellular proteins. In addition, peroxynitrite targets DNA, leading to chain breaks and DNA base oxidation. Thus, the release of free radicals damages cardiac muscle. Compounds of the invention (e.g., BH4) that reduce NOS uncoupling prevent the formation of reactive oxygen species and subsequent compounds such as ONOO⁻. Such compounds are particularly useful when combined with free radical scavengers or anti-oxidants.

Agents that Prevent or Treat Cardiac Hypertrophy and Dilation

Tetrahydrobiopterin (BH4) is one exemplary agent that modulates NOS. BH4 is a required co-factor for normal NOS synthesis of NO. In the absence of BH4, NOS enzymes are uncoupled, and generate substantial quantities of superoxide. Reduced levels of BH4 occur in clinically relevant disease conditions such as cardiac failure, hypertrophy, and vascular diseases—hypertension and atherosclerosis. A decline in BH4 also results in NOS3 uncoupling. Replacement therapy with BH4 thereby shifts the balance of NOS3 activity away from the production of reactive oxygen species and towards the production of nitric oxide. BH4 has been used for the treatment of the inherited metabolic disorder, atypical hyperphenylalaninemia, which is caused by a deficiency of the enzyme phenylalanine hydroxylase (PAH). BH4 serves as a cofactor for PAH, and though replacement therapy by BH4 does not directly resolve the deficiency of PAH, in heterozygous deficient subjects, it helps to favor the reaction despite the lack of normal PAH levels. BH4 (2-5 mg/kg/day) is administered orally. Commercial preparations of BH4 are available, such as PHENOPTIN (oral tetrahydrobiopterin) (BioMarin, Novato, Calif.), BIOPTEN, (Sapropterin Hydrochloride) (Suntory Ltd., Daiichi Suntory Pharma Co., Ltd., Japan). BH4 dosage generally range from 2-5 mg/kg/day to 10-20 mg/kg/day. BH4 dosage may be titrated to determine effective maintenance doses at which serum phenylalanine levels are maintained in the normal range.

In vivo BH4 is synthesized de novo from guanosine 5′-triphosphate (GTP) by the primary enzyme GTP cyclo-hydrolase. BH4 can also be generated by salvage pathways from 7,8-dihydrobiopterin (BH2). BH4 is oxidized to 7,8-dihydrobiopterin, and BH4 homeostasis is maintained by BH4 synthesis and oxidation. Anti-oxidants, such as Vitamin C and folate, enhance BH4 availability by scavenging reactive oxygen species, chemical stabilization, and in the case of folate, by enhancing the salvage pathway from BH2. Such agents are likely to be useful for the treatment of cardiac diseases or disorders. Exemplary anti-oxidants include vitamin C, super oxide dismutase (SOD), n-acetyl cysteine, reduced glutathione, vitamin E, allopurinol and oxypurinol, and TEMPOL. Agents that enhance BH4 efficacy are also useful alone or in combination with BH4. For example, agents that increase salvage of BH4 are useful in the methods of the invention; as are agents that increase levels of cGMP, including soluble guanylate cyclase activators (e.g., YC-1 (Wu et al., Br J Pharmacol. 116: 1973-1978, 1995), BAY 58-2667 (Garner-Hamrick et al., BMC Pharmacology 5(Suppl 1):P20, 2005) or BAY 41-2272 (Deruelle et al., Am J Physiol Lung Cell Mol Physiol 288: L727-L733, 2005), BAY-41-8543 (Stasch et al., Br J Pharmacol. January; 135(2):344-55, 2002). Accordingly, agents that enhance BH4 efficacy, stabilization, salvage, or that increase cGMP levels may be used alone or in combination with BH4 in the methods of the invention for the treatment of cardiac diseases and disorders. Also useful in combination with BH4 are PDE5 inhibitors (e.g., sildenafil, tadalafil, vardenafil). PDE5 is a cGMP-selective phosphodiesterase, and its inhibition increases cGMP levels.

### PDE5 Inhibitors

PDE5 is expressed in systemic and pulmonary arterial and venous smooth muscle cells—particularly in the corpus cavernosum. In light of this expression, PDE5 inhibitors were initially of interest for their vasodilatory effects. Sildenafil, for example, was first studied as an anti-anginal medication in anticipation of its capacity to dilate coronary arteries. Early clinical studies of sildenafil for the treatment of angina, however, were disappointing, as its impact on arterial vasodilation was very modest. These clinical studies did lead to the finding that erectile function was improved as a common side effect of sildenafil administration. Sildenafil enhances an erection by decreasing the breakdown of cGMP and thus prolonging the vasodilatory effects induced in the penile circulation by nitric oxide in response to sexual stimulation. This same cyclic nucleotide signaling pathway mediates the smooth-muscle relaxing effects of nitric oxide necessary for normal erectile function. Down-regulation of this pathway is central to the pathophysiology of many forms of erectile dysfunction.

Sildenafil is selective for PDE5. PDE5 plays an important role in hearts subjected to stress, and PDE5A inhibition prevents and reverses morphological, cellular, and molecular remodeling in hearts that are subject to stress related to pressure-overload, neurohormonal stress, myocardial infarction, or volume-overload. See, for example, Takimoto et al., Nat Med. 11(2):214-22, 2005. Surprisingly, the therapeutic effects of PDE5 inhibitors on heart function, left heart function, hypertrophy, and molecular and cellular remodeling are achieved in the complete absence of any change in the load imposed on the heart.

PDE5 inhibitors are known in the art, and include, but are not limited to, sildenafil (Compound 1), vardenafil (Compound 2), tadalafil (Compound 3), EMD 360527, DA 8159, or analogs thereof, or any other compound that inhibits cGMP hydrolysis by phosphodiesterase-5 (PDE5). See also U.S. Pat. Nos. 6,916,927, 6,911,542, 6,903,099, 6,878,711, 6,872,721, 6,858,620, 6,825,197, 6,774,128, 6,723,719, 6,699,870, 6,670,366, 5,859,006 and 5,250,534. Other PDE5 inhibitors useful in the methods of the invention are described in WO 03/063875; WO 03/1012761 WO 2004/037183, and WO 98/38168. All of these patents and patent applications are incorporated herein by reference in their entirety.

Sildenafil is commercially available in three dosages of 25, 50, or 100 mg and has an IC₅₀ of approximately 10 nM. Effective plasma concentrations are between 1 nM and 250 nM, where the bottom of the range is any integer between 1 and 249; and the top of the range is any integer between 2 nM and 250 nM. Preferably, an effective plasma concentration is between 5 nM and 100 nM, more preferably it is between 10 nM and 50 nM (e.g., 15 nM, 20 nM, 25 nM, 30 nM, 40 nM, or 45 nM).

Tadalafil is commercially available in three dosages of 5, 10, or 20 mg and has an IC₅₀ of approximately 1 nM. Following oral administration of a 20 mg dose of tadalafil to healthy subjects, tadalafil is rapidly absorbed with the peak plasma concentration of 378 ng/ml occurring two hours post-dose. Preferably an effective plasma concentration is between 5 nM and 100 nM, more preferably it is between 10 nM and 50 nM (e.g., 15 nM, 20 nM, 25 nM, 30 nM, 40 nM, or 45 nM). Tadalafil has a relative large apparent volume of distribution (Vd/F) of 62.6 L, and a low apparent oral clearance (CL/F) of 2.48 L/h. As a result, the mean elimination half-life of tadalafil is about 17.5 h, which is substantially longer than that of sildenafil or vardenafil.

Vardenafil is commercially available in three dosages of 5 mg, 10 mg, and 20 mg and has an IC₅₀ of 0.7 nM. Effective plasma concentrations of vardenafil are between 0.1 and 5.0 nM.

The skilled artisan appreciates that any compound that reduces the activity of PDE5 is useful in the methods of the invention. Other exemplary compounds useful in the methods of the invention include UK-343,664 (Walker et al., Xenobiotica, 31: 651-664), UK-427,387, UK-357903 [1-ethyl-4-{3-[3-ethyl-6,7-dihydro-7-oxo-2-(2-pyridylmethyl)-2H-pyrazolo[4,3-d]pyrimidin-5-yl]-2-(2-methoxyethoxy)-5-pyridylsulphonyl}piperazine] (Gardiner et al. J Pharmacol Exp Ther. 2005; 312: 265-271), UK-371800 (Pfizer), UK-313794 (Pfizer) and UK-343664 (Abel et al., Xenobiotica. 2001 31:665-76); TA-1790 from Tanabe Seiyaku; CP-248, CP-461 and exisulind (Deguchi et al., Molecular Cancer Therapeutics 803-809, 2002), which are available from Osi Pharmaceuticals; pyrazolinone; EMD82639 (4-(4-[2-ethyl-phenylamino)-methylene]-3-methyl-5-oxo-4,5-di-hydro-pyrazol-1-yl)-benzoic acid (Senzaki et al., FASEB Journal. 2001; 15:1718-1726); [7-(3-Chloro-4-methoxy-benzylamino)-1-methyl-3-propyl-1H-pyrazolo[4,3-d]pyrimidin-5-ylmethoxy]-acetic acid (EMD360527), 4-[4-(3-Chloro-4-methoxy-benzylamino)-benzo[4,5]thieno[2,3-d]-pyrimidin-2-yl]-cyclohexanecarboxylic acid, ethanolamin salt (EMD221829) and 5-[4-(3-Chloro4-methoxy-benzylamino)-5,6,7,8-tetrahydro-benzo[4,5]thieno[2,3-d]pyrimidin-2-yl]-pentanoic acid (EMD171827), which are commercially available from Merck KgaA (Darmstadt, Del.) and are described, for example, in Scutt et al. (BMC Pharmacol. 2004; 4: 10); 3-(1-Methyl-7-oxo-3-propyl-6,7-dihydro-1H-pyrazolo-[4,3-d]pyrimidin-5-yl)-N-[2-(1-methylpyrrolidin-2-yl)ethyl]-4-propoxybenzenesulfonamide (DA-8259); E4021 (Dukarm et al., Am. J. Respir. Crit. Care Med., 1999, 160:858-865); pentoxifylline and FR22934 (Fujisawa).

### Cardiovascular Function

Cardiac conditions, such as cardiac hypertrophy, reduced systolic function, reduced diastolic function, maladaptive hypertrophy, heart failure with preserved systolic function, diastolic heart failure, hypertensive heart disease, aortic and mitral valve disease, pulmonary valve disease, hypertrophic cardiomyopathy (e.g., hypertrophic cardiomyopathy originating from a genetic or a secondary cause), post ischemic and post-infarction cardiac remodeling and cardiac failure, are associated with maladaptive cardiac alterations, cardiac chamber, cellular, and molecular remodeling. Compositions of the invention may be used to enhance cardiac function in a subject having reduced cardiac function. Desirably, cardiac function is increased by at least 5%, 10% or 20%, or even by as much as 25%, 50% or 75%. Most advantageously, cardiac function is enhanced or cardiac damage, including hypertrophy or dilation, is reversed, such that the cardiac function is substantially normal (e.g., 85%, 90%, 95%, or 100% of the cardiac function of a healthy control subject). Alternatively, such assays are used to monitor the condition of a subject prior to, during, or following treatment with tetrahydrobiopterin (BH4) or with a combination of the invention that includes BH4 and at least one of an anti-oxidant, folate, a PDE5A inhibitor, and a soluble guanylate cyclase activator (e.g., YC-1, BAY 58-2667, BAY 41-2272, or BAY-41-8543). Treatments that increase cardiac function are useful in the methods of the invention.

Any number of standard methods are available for assaying cardiovascular function.

Preferably, cardiovascular function in a subject (e.g., a human) is assessed using non-invasive means, such as measuring net cardiac ejection (ejection fraction, fractional shortening, and ventricular end-systolic volume) by an imaging method such echocardiography, nuclear or radiocontrast ventriculography, or magnetic resonance imaging, and systolic tissue velocity as measured by tissue Doppler imaging. Systolic contractility can also be measured non-invasively using blood pressure measurements combined with assessment of heart outflow (to assess power), or with volumes (to assess peak muscle stiffening). Measures of cardiovascular diastolic function include ventricular compliance, which is typically measured by the simultaneous measurement of pressure and volume, early diastolic left ventricular filling rate and relaxation rate (can be assessed from echoDoppler measurements). Other measures of cardiac function include myocardial contractility, resting stroke volume, resting heart rate, resting cardiac index (cardiac output per unit of time [L/minute], measured while seated and divided by body surface area [m²])) total aerobic capacity, cardiovascular performance during exercise, peak exercise capacity, peak oxygen (O₂) consumption, or by any other method known in the art or described herein. Measures of vascular function include determination of total ventricular afterload, which depends on a number of factors, including peripheral vascular resistance, aortic impedance, arterial compliance, wave reflections, and aortic pulse wave velocity,

Methods for assaying cardiovascular function include any one or more of the following: Doppler echocardiography, 2-dimensional echo-Doppler imaging, pulse-wave Doppler, continuous wave Doppler, oscillometric arm cuff, tissue Doppler imaging, cardiac catheterization, magnetic resonance imaging, positron emission tomography, chest X-ray, X-ray contrast ventriculography, nuclear imaging ventriculography, computed tomography imaging, rapid spiral computerized tomographic imaging, 3-D echocardiography, invasive cardiac pressures, invasive cardiac flows, invasive cardiac pressure-volume loops (conductance catheter), non-invasive cardiac pressure-volume loops.

### Pharmaceutical Compositions

The present invention features pharmaceutical preparations for the treatment of cardiac indications, where the pharmaceutical preparation comprises a compound (e.g., an NOS or NOS3 modulator) that reduces NOS uncoupling or that enhances NO production together with a pharmaceutically acceptable carriers. In one example, tetrahydrobiopterin (BH4) or a combination of the invention (e.g., BH4 and at least one of an anti-oxidant, folate, a PDE5A inhibitor, and a soluble guanylate cyclase activator) is provided in a carrier, where the compounds provide for the treatment of virtually any cardiac indication characterized by the hypertrophic morphological, cellular, or molecular remodeling of a cardiac tissue. Pharmaceutical preparations of the invention have both therapeutic and prophylactic applications. In one embodiment, a pharmaceutical composition includes an effective amount of an NOS3 modulator, such as BH4. The compositions should be sterile and contain a therapeutically effective amount of a PDE5 inhibitor in a unit of weight or volume suitable for administration to a subject (e.g., a human patient). The compositions and combinations of the invention can be part of a pharmaceutical pack, where the PDE5 inhibitor is present in individual dosage amounts.

Pharmaceutical compositions of the invention to be used for prophylactic or therapeutic administration should be sterile. Sterility is readily accomplished by filtration through sterile filtration membranes (e.g., 0.2 μm membranes), by gamma irradiation, or any other suitable means known to those skilled in the art. Therapeutic compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution.

An NOS3 modulator may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with an NOS3 modulator, such as BH4, of the present invention, and with each other, in a manner such that there is no interaction that would substantially impair the desired pharmaceutical efficacy.

Compounds of the present invention can be contained in a pharmaceutically acceptable excipient. The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having a cardiac disease or disorder associated with hypertrophic morphological, cellular, or molecular remodeling, an effective amount is sufficient to prevent, reduce, stabilize, or reverse an alteration associated with cardiac hypertrophy. With respect to a subject having a cardiac disease or disorder, an effective amount is an amount sufficient to stabilize, slow, or reduce a symptom associated with the cardiac condition.

Generally, doses of the compounds of the present invention would be from about 0.01 mg/kg per day to about 1000 mg/kg per day. Typically, 1-10 mg/kg/day BH4 is administered orally (e.g., 1, 2, 3, 4, 5, 6, 7, 8, 9, or 10). Higher doses are generally required for treatment of phenylketonuria (PKU), however lower doses would likely be required for the purposes of restoring NOS function and its resulting impact on cardiac indications and/or the efficacy of adjunctive agents (e.g. PDE5 inhibitor, anti-oxidant, soluble guanylate cyclase activator) for a combined effect on cardiac indications.

In one embodiment, BH4 is administered in combination with a PDE5 inhibitor. Typically 25, 50, 75, 100, 125, 150 or 200 mg of a PDE5 inhibitor, such as sildenafil, is administered to a subject. Preferably, 100 mg of a PDE5 inhibitor is administered. Desirably, the PDE5 inhibitor is administered in an amount sufficient to achieve a peak concentration of 10, 25, 50, 75, or 100 nM in plasma. Preferably, the peak concentration is 50 nM. Effective doses range from 0.1 nM to 200 nM, where the bottom of the range is any integer between 1 and 199, and the top of the range is any integer between 2 and 200. Desirably, an effective dose results in a free plasma PDE5 inhibitor concentration ranging from 10-50 mM; but it can be as much as 200 nM or as low as 1-2 mM. Exemplary concentrations include 0.1, 1, 5, 10, 20, 25, 30, 40, or 50 nM. It is expected that doses ranging from about 5 to about 2000 mg/kg will be suitable—depending on the specific PDE5a inhibitor used. Lower doses will result from certain forms of administration, such as intravenous administration and pharmaceutical. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of a composition of the present invention.

A variety of administration routes are available. The methods of the invention, generally speaking, may be practiced using any mode of administration that is medically acceptable, meaning any mode that produces effective levels of the active compounds without causing clinically unacceptable adverse effects. In one preferred embodiment, a composition of the invention is administered orally. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g., tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising a compound of the present invention can contain multivalent metal ions, such as calcium ions, magnesium ions and/or manganese ions. Any multivalent metal ion that helps stabilizes the composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the compound contained therein. Preferably, the non-aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000.

Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly(2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μM, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into a mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

### Methods of Treatment

In one embodiment, the present invention provides a method of modulating NOS activity in the heart of a subject comprising the step of administering to the subject an effective amount of an NOS3 modulator, such as BH4, alone or in combination with an anti-S oxidant (e.g. vitamin C), folate, an activator of soluble guanylate cyclase (sGC), or a PDE5 inhibitor, preferably as part of a composition additionally comprising a pharmaceutically acceptable carrier. Preferably this method is employed to treat a subject suffering from or susceptible to a cardiac condition selected from cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure. Other embodiments include any of the methods herein wherein the subject is identified as in need of the indicated treatment.

In a second embodiment, the present invention provides a method of directly stimulating the distal target of NOS activity, soluble guanylate cyclase (sGC) in combination with an inhibitor of PDE5.

Another aspect of the invention is the use of an NOS3 modulator in the manufacture of a medicament for enhancing cardiac function or reducing morphological, cellular, or molecular remodeling in a subject. Preferably, the medicament is used for treatment or prevention in a subject of a disease, disorder or symptom set forth above. Such medicaments include, for example, BH4 or a combination of BH4 and at least one of an anti-oxidant, folate, a PDE5A inhibitor, and a soluble guanylate cyclase activator.

### Kits

The invention provides kits for the treatment or prevention of a cardiac condition associated with cardiac hypertrophy, including morphological, cellular, or molecular remodeling. In one embodiment, the kit includes a pharmaceutical pack comprising an effective amount of an NOS modulator, such as BH4. In other embodiments, the kit provides BH4 in combination with at least one of an anti-oxidant, folate, a PDE5A inhibitor, and a soluble guanylate cyclase activator. Preferably, the compositions are present in unit dosage form. In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention or combinations thereof are provided together with instructions for administering them to a subject having or at risk of developing a cardiac condition associated with hypertrophy. The instructions will generally include information about the use of the compounds for the treatment or prevention of a cardiac condition associated with hypertrophy. In other embodiments, the instructions include at least one of the following: description of the compound or combination of compounds; dosage schedule and administration for treatment of a cardiac condition or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The following examples are provided to illustrate the invention, not to limit it. Those skilled in the art will understand that the specific constructions provided below may be changed in numerous ways, consistent with the above described invention while retaining the critical properties of the compounds or combinations thereof.

### EXAMPLES

NOS3 is the dominant isoform of nitric oxide synthase present in the endothelium as well as in cardiac myocytes, where it is an important regulator of adrenergic, muscarinic, and rate-mediated reserve. Active NOS3 is a homodimer that generates NO and L-citrulline from L-arginine. NOS can be converted to a reactive oxygen species generator when exposed to oxidant stress, including peroxinitrite (ONOO⁻), or deprived of its reducing cofactor tetrahydrobiopterin (BH4) or substrate L-arginine, NOS3 uncouples to the monomeric form that generates O₂⁻ rather than NO.

As described in more detail below, chronic transverse aortic constriction in control mice induced marked cardiac hypertrophy, dilation and dysfunction. Mice lacking NOS3 displayed modest and concentric hypertrophy to transverse aortic constriction with preserved function. NOS3^{-/-}-transverse aortic constriction hearts developed less fibrosis, myocyte hypertrophy and fetal gene re-expression (B-natriuretic peptide, α-skeletal actin). Reactive oxygen species, nitrotyrosine (NT) and gelatinase (MMP-2 and MMP-9) zymogen activity markedly increased in control-transverse aortic constriction, but not in NOS3^{-/-}-transverse aortic constriction hearts. Transverse aortic constriction induced NOS3 uncoupling in the heart, reflected by reduced NOS3 dimer and tetrahydrobiopterin (BH4), increased NOS3-dependent generation of reactive oxygen species, and lowered Ca²⁺-dependent NOS activity. Co-treatment with BH4 prevented NOS3 uncoupling and inhibited reactive oxygen species, resulting in concentric non-dilated hypertrophy. Mice given the anti-oxidant tetrahydroneopterin as a control did not display changes in transverse aortic constriction response. Thus, pressure-overload triggers NOS3 uncoupling as a prominent source of myocardium reactive oxygen species that contribute to dilation/remodeling and cardiac dysfunction. Reversal of this process by BH4 suggests a potential treatment to ameliorate the pathophysiology of chronic pressure-induced hypertrophy.

### Example 1

### Lack of NOS3 Ameliorated Cardiac and Myocyte Hypertrophy, Dilation, and Fibrosis Due to Transverse Aortic Constriction

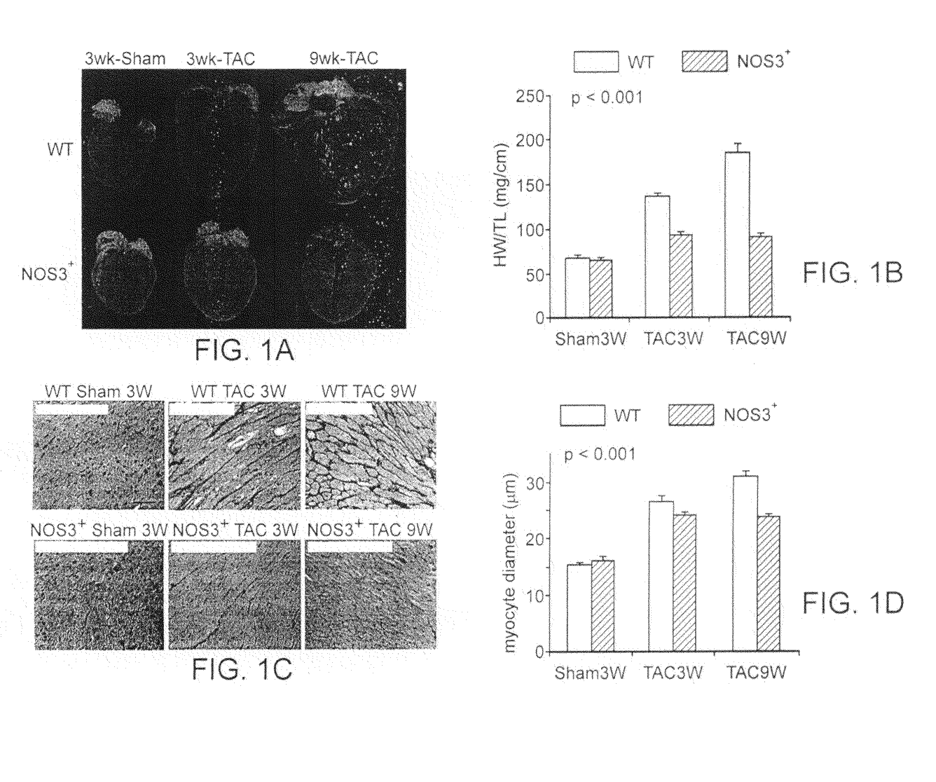

In control wild-type mice, heart weight normalized to tibia length (HW/TL) increased 100% after 3-weeks of pressure-overload induced by transverse aortic constriction (TAC), and by 175% after 9 weeks (FIGS. 1A and 1B). This was accompanied by near doubling of myocyte diameter (at 9 weeks) and increased interstitial fibrosis (FIGS. 1C and 1D). Collagen fraction rose 0.1±0.1% to 4.0±0.8% after 9 weeks TAC (p<0.005), increasing further with prolonged TAC. In contrast, the hypertrophic response to TAC in NOS3^{-/-}-hearts was far more modest, with an increase in myocyte size nearly half that of wild-type after 9-weeks of TAC, and chamber size was smaller (i.e. concentric hypertrophy). Basal collagen fraction was somewhat elevated in NOS3^{-/-}-hearts although still low (1.5±0.7%), but this did not change with TAC (e.g. 1.9±0.9% 3-weeks, similar results at 9-weeks). Importantly, NOS3^{-/-} and wild-type hearts had similar heart mass and myocyte size at baseline, and the rise in ventricular systolic pressure and ventricular afterload (arterial elastance, Eₐ) induced by TAC was similar or greater (at 9 weeks) in NOS3^{-/-} hearts over wild-type controls.

### Example 2

### Lack of NOS3 Ameliorates Left Ventricular Dysfunction Induced by Transverse Aortic Constriction

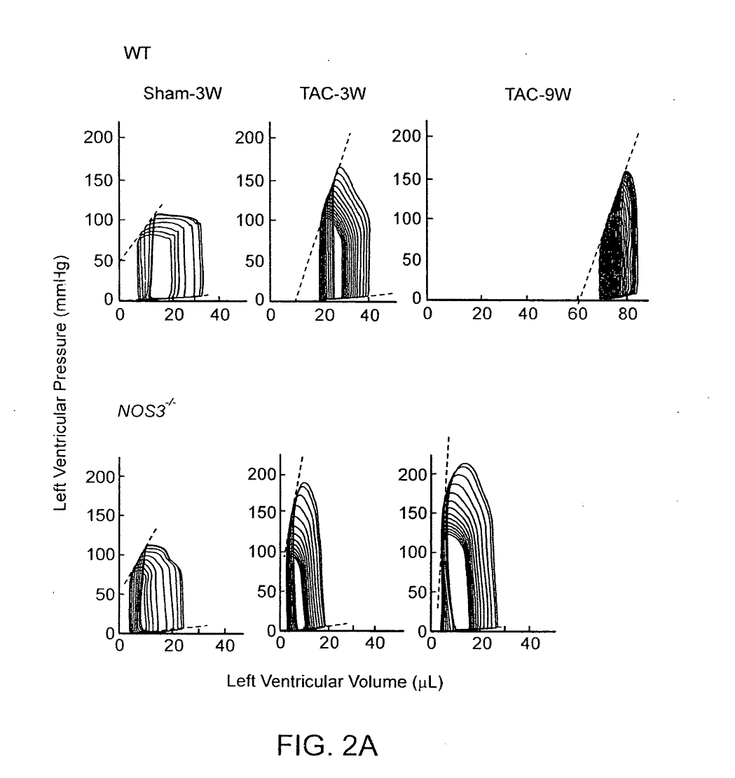

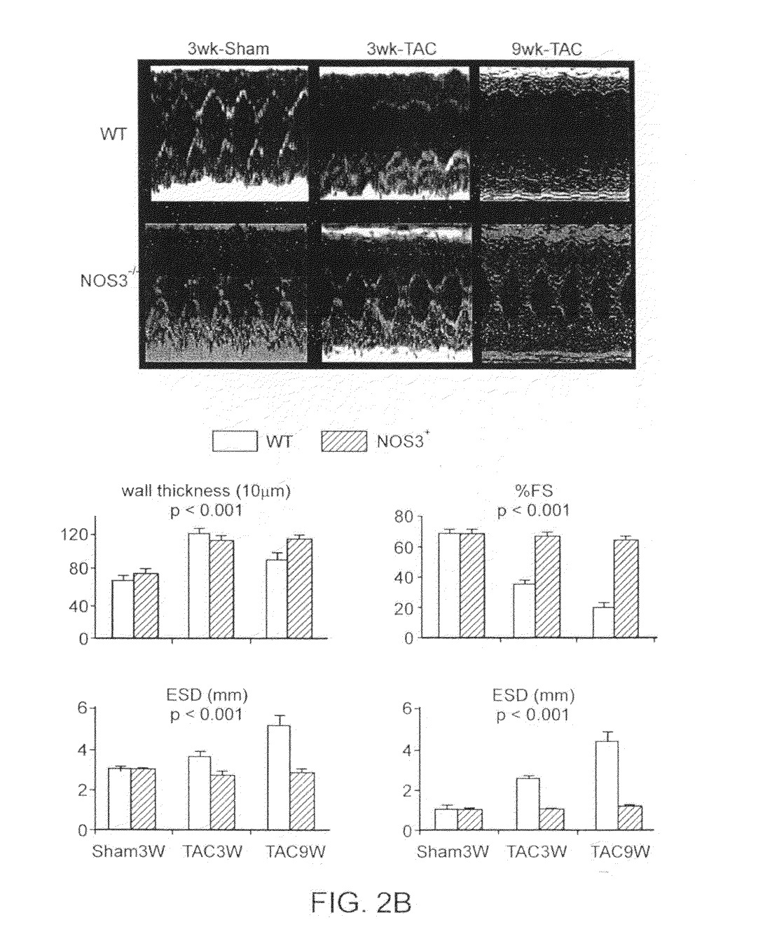

Marked disparities of in vivo cardiac function were observed between wild-type and NOS3^{-/-} animals exposed to TAC (FIGS. 2A and 2B, Table 1), with wild-type-TAC displaying progressive cardiac decompensation while NOS3^{-/-} hearts had preserved or even enhanced function.

TABLE 1
TAC induced changes in cardiac morphology and left ventricular function

| | Genotype | Baseline | TAC3W | TAC9W | ANOVA |
|---|---|---|---|---|---|
| Body Weight (gm) | WT | 27.6 ± 0.6 | 27.2 ± 0.3 | 26.5 ± 0.5 | a, c |
| | NOS3^{-/-} | 26.1 ± 0.3 | 25.9 ± 0.2* | 29.9 ± 0.2* | |
| Heart Weight (mg) | WT | 122.9 ± 4.0 | 241.1 ± 6.8 | 344.0 ± 20.0 | a, b, c |
| | NOS3^{-/-} | 116.1 ± 2.4 | 165.3 ± 5.3* | 166.3 ± 4.0* | |
| Heart Rate (min⁻¹) | WT | 522.6 ± 13.7 | 520.0 ± 13.0 | 542 ± 8.6 | a |
| | NOS3^{-/-} | 499 ± 5.0 | 500.3 ± 18.3 | 589.1 ± 18.0 | |
| LV Systolic Pressure | WT | 107.0 ± 2.2 | 179.9 ± 3.1 | 168.0 ± 5.1 | a, b, c |
| (mmHg) | NOS3^{-/-} | 120.7 ± 3.8* | 182.8 ± 3.5 | 212.4 ± 6.7* | |
| LV End Diastolic | WT | 5.4 ± 0.6 | 7.1 ± 1.4 | 5.1 ± 1.1 | |
| Pressure (mmHg) | NOS3^{-/-} | 7.1 ± 0.4 | 7.5 ± 0.7 | 7.9 ± 1.4 | |
| Effective Arterial | WT | 5.5 ± 0.4 | 10.4 ± 0.5 | 11.9 ± 0.4 | a, b |
| Elastance (mmHg/μl) | NOS3^{-/-} | 6.4 ± 0.4 | 12.4 ± 0.6* | 19.1 ± 3.9 | |
| LV End Diastolic | WT | 29.0 ± 2.0 | 38.8 ± 3.4 | 73.0 ± 10.3 | a, b, c |
| Volume (μL) | NOS3^{-/-} | 34.2 ± 4.3 | 19.9 ± 1.7* | 17.3 ± 3.0* | |
| LV End Systolic | WT | 10.2 ± 1.0 | 23.3 ± 3.3 | 59.0 ± 10.5 | a, b, c |
| Volume (μL) | NOS3^{-/-} | 15.5 ± 4.6 | 6.7 ± 1.4* | 5.9 ± 1.6* | |
| Ejection Fraction (%) | WT | 65.1 ± 2.1 | 41.3 ± 3.6 | 20.0 ± 3.1 | a, b, c |
| | NOS3^{-/-} | 57.2 ± 8.1 | 67.7 ± 3.7* | 65.7 ± 9.3* | |
| dPdtₘₓ (mmHg/s) | WT | 13368 ± 370 | 12602 ± 620 | 10004 ± 596 | a, d, c |
| | NOS3^{-/-} | 10705 ± 991* | 11963 ± 556 | 18232 ± 1146* | |
| Peak Power Index | WT | 31.6 ± 0.9 | 41.5 ± 1.9 | 21.4 ± 6.1 | a, b, c |
| (mmHg/s) | NOS3^{-/-} | 26.8 ± 3.2 | 60.0 ± 3.7* | 59.3 ± 8.5* | |
| Normalized Ees | WT | 37.9 ± 5.8 | 70.2 ± 13.4 | 21.6 ± 4.5 | a, b, c |
| (mmHg/μL/g) | NOS3^{-/-} | 34.8 ± 5.4 | 158.4 ± 34.6* | 154.9 ± 19.3* | |
| dPdtₘₙ (mmHg/s) | WT | −10728 ± 236 | −10508 ± 500 | −8462 ± 268 | a, b, c |
| | NOS3^{-/-} | −10470 ± 409 | −11822 ± 422 | −18293 ± 436* | |
| Peak Filling Rate/EDV | WT | 37.1 ± 5.6 | 24.4 ± 1.4 | 15.1 ± 3.1 | d |
| (sec⁻¹) | NOS3^{-/-} | 33.5 ± 2.7 | 43.7 ± 6.1* | 35.4 ± 10.1 | |
| Tau (msec) | WT | 4.1 ± 0.2 | 5.0 ± 0.2 | 6.3 ± 0.2 | b, c |
| | NOS3^{-/-} | 5.0 ± 0.1* | 5.0 ± 0.2 | 3.7 ± 0.2* | |

Data are mean ± sem. Controls are 3 week sham operated mice. For BW and HW: n = 8, 18, and 10 for WT and n = 6, 18, and 11 for NOS3^{-/-} for control, TAC3W, TAC9W, respectively in each genotype. For hemodynamic measures: n = 5, 6, and 3 for WT and n = 4, 6, and 4 for NOS3^{-/-} for control, TAC3W, TAC9W, respectively in each genotype. Ees - left ventricular end-systolic elastance. Peak power index = maximal LV power/EDV.
*p < 0.05 vs WT (unpaired t-test) at each time point.
(a-d) - two way ANOVA results; a: p < 0.01 for time effect; b: p < 0.01 for genotype effect; d: p < 0.05 for genotype effect; and c: p < 0.01 for time-genotype interaction.

In wild-type hearts, TAC induced a rightward shift of the left ventricular pressure-volume (PV) loops (FIG. 2A) and end-systolic and end-diastolic PV relations reflecting remodeling. This was quite marked after 9-weeks TAC (e.g. 5-fold increase in end-systolic volume). In contrast, PV loops and relations of NOS3^{-/-} mice exposed to TAC shifted leftward with smaller end-diastolic but also end-systolic chamber volumes. Net stroke volume and cardiac output declined with TAC in both groups, but these changes were similar in both genotypes. Contractility was determined by end-systolic elastance (slope of relation at upper left corners of each loop-set), dP/dtₘₐₓ, and maximal power index. All rose with sustained TAC in NOS3^{-/-} animals but declined significantly in wild-type-TAC mice (at 9 weeks). Diastolic function showed analogous disparities, with rate of pressure decline slowed by TAC in wild-type mice, but unchanged (at 3 weeks) or slightly enhanced (at 9 weeks) in NOS3^{-/-} mice. The disparities in chamber volumes were further confirmed by echocardiography in conscious mice (FIG. 2B). This analysis also demonstrated that increased wall thickness was similar between genotypes at 3 weeks; thus, the major disparity was related to concentric versus eccentric (dilative) hypertrophic remodeling.

### Example 3

### Differential Response in Fetal Gene Expression

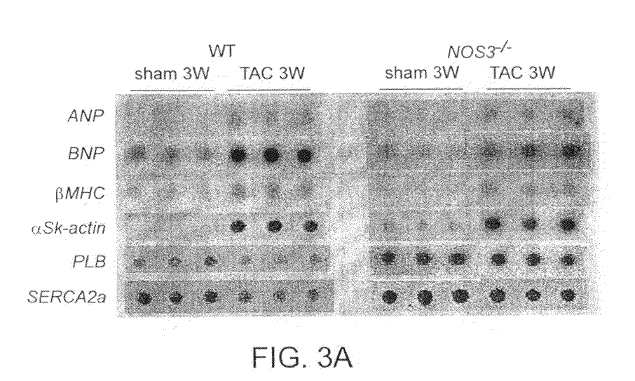

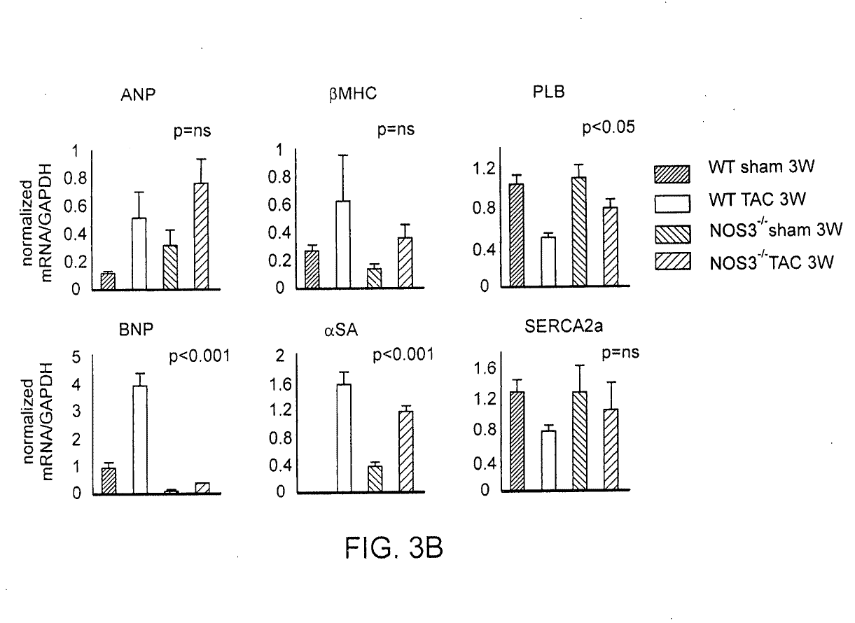

In wild-type controls, TAC (3-weeks) triggered fetal gene re-expression, increasing mRNA levels for type A and B natriuretic peptides (NP), β-myosin heavy chain (β-MHC), α-skeletal actin (α-SA), and reducing expression of phospholamban (PLB) and sarcoplasmic reticulum Ca²⁺ ATPase (SERCA2a) (all p<0.01; FIGS. 3A and 3B). In NOS3^{-/-} hearts, TAC induced similar changes in some of these genes but not in others. In particular, type B-NP and α-SA were enhanced to a lesser extent, whereas type A-NP and β-MHC were similarly elevated in both genotypes. PLB declined less with TAC in NOS3^{-/-} (p<0.05), while a directionally similar disparity in SERCA2a expression fell short of significance. Thus, different hypertrophy phenotypes between groups were accompanied by selective fetal gene re-expression.

### Example 4

### Reactive Oxygen Species Generation is Blunted in NOS3^{-/-} Pressure-Loaded Hearts

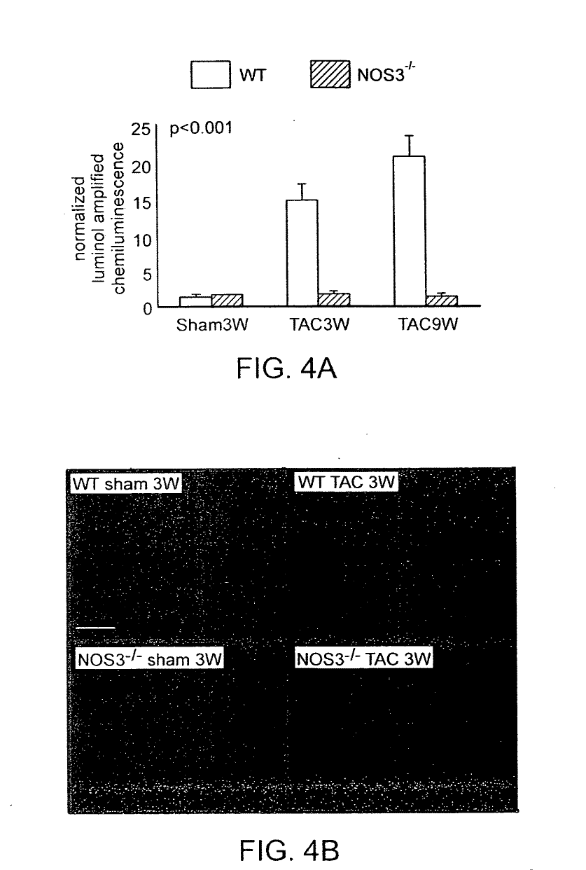

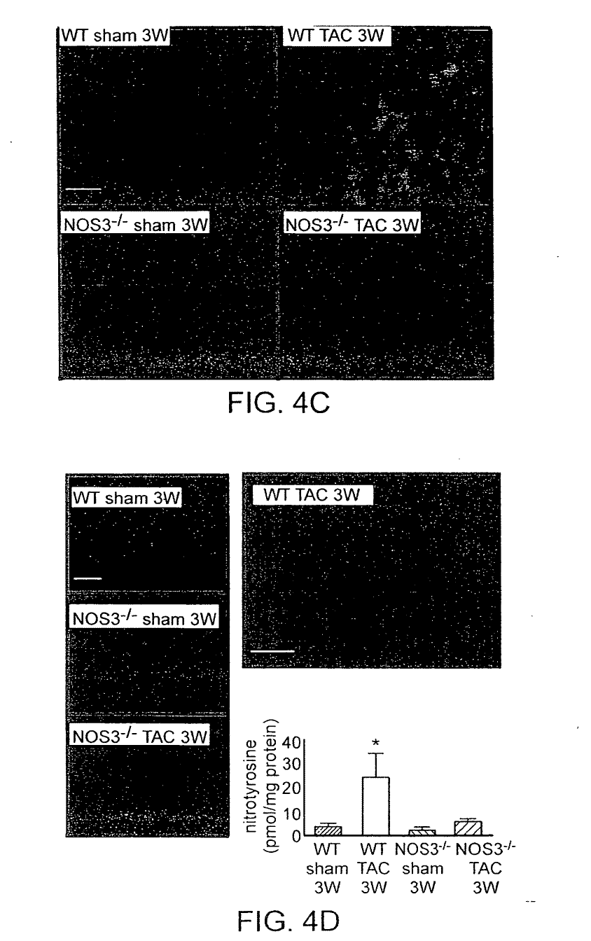

To determine whether TAC induced myocardial reactive oxygen species, and if this differed between wild-type and NOS3^{-/-} hearts, left ventricular myocardial O₂⁻ production was assessed by luminol chemiluminescence increased significantly in wild-type-TAC hearts over sham-controls, whereas this was not observed in NOS3^{-/-}-TAC hearts (FIG. 4A). Similar disparities were observed in dihydroethidium (DHE) and dichlorofluorescein (DCF) stained left ventricular myocardium (FIGS. 4B and 4C). NO interacts with O₂⁻ to form ONOO⁻ a potent oxidant whose presence may be indirectly reflected by an increase in nitrotyrosine formation. Nitrotyrosine immunostaining increased substantially in wild-type-TAC hearts (FIG. 4D) yet was minimal in NOS3^{-/-}-TAC hearts. This was quantitatively confirmed by separate ELISA analysis for nitrotyrosine (bar graph, FIG. 4D).

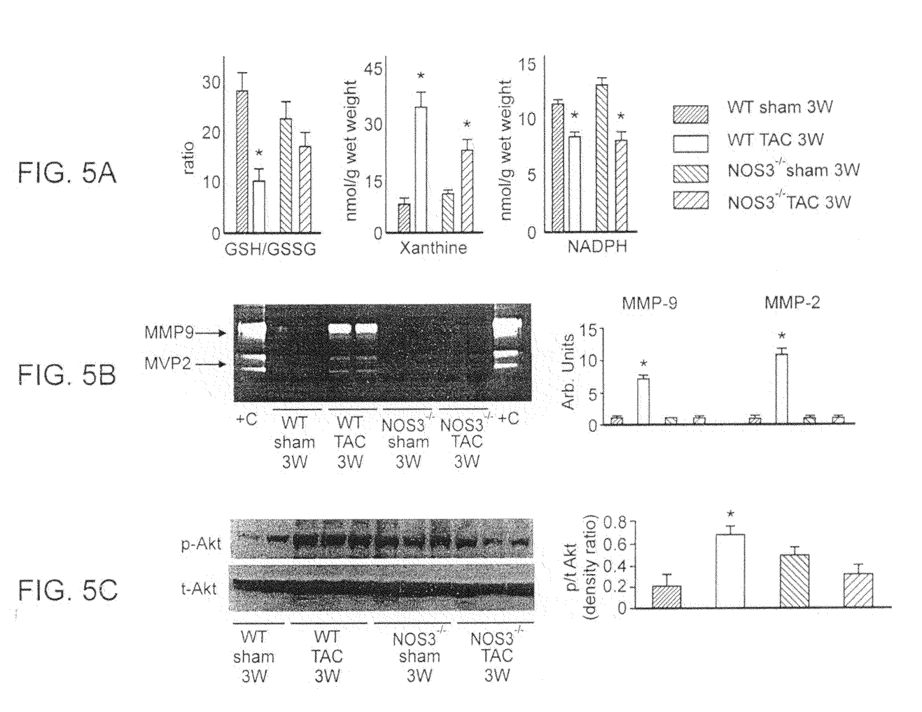

To further probe for disparities in oxidative stress, the ratio of reduced to oxidized glutathione (GSH/GSSG) was assessed by high-performance liquid chromatography (HPLC). This assay showed that it declined significantly in wild-type-TAC yet was little altered in NOS3^{-/-}-TAC heart (FIG. 5A). Purine catabolites related to xanthine oxidase activity (xanthine, uric acid) rose significantly with TAC in both groups (FIG. 5A shows xanthine data), although somewhat less in NOS3^{-/-}. Some oxidant stress was also reflected by a fall in NADPH in both groups (FIG. 5A), although NADPH/NADP ratio did not significantly change.

### Example 5

### MMP-2, MMP-9, and p-Akt Increases with Pressure-Load are Blunted in Mice Lacking NOS3

Since the major disparity in TAC response between genotypes was related to chamber remodeling, cardiac gelatinases MMP-2 and MMP-9 which are activated by reactive oxygen species and potent contributors to cardiac dilation^{1,2} were examined. Both MMP-2 and MMP-9 zymogram gel-lysis was minimal in sham controls (both genotypes). While wild-type-TAC displayed markedly increased gel lysis for both MMPs, this was lacking in NOS3^{-/-}-TAC hearts (FIG. 5B, FIG. 5C).

Reactive oxygen species can inactive the tumor suppressor PTEN, a phosphatidylinositol (3,4,5) trisphosphate 3 phosphatase, resulting in increased activity of PI3-kinase and thus phosphorylation of Akt kinase³. Increased phosphorylated-Akt (p-Akt) is associated with cardiac hypertrophy^{4,5} and mice overexpressing Akt develop hypertrophy⁶. It was therefore tested whether Akt phosphorylation was differentially altered by TAC in wild-type versus NOS3^{/-} mice. Both total and serine 379 phosphorylated Akt (s379/p-Akt) increased in wild-type-TAC, with a disproportionate rise in p-Akt (>6-fold). In contrast, TAC did not significantly alter Akt in NOS3^{-/-}-TAC hearts (FIG. 5C).

### Example 6

### NOS3 Uncoupling in Wild-Type-TAC Hearts

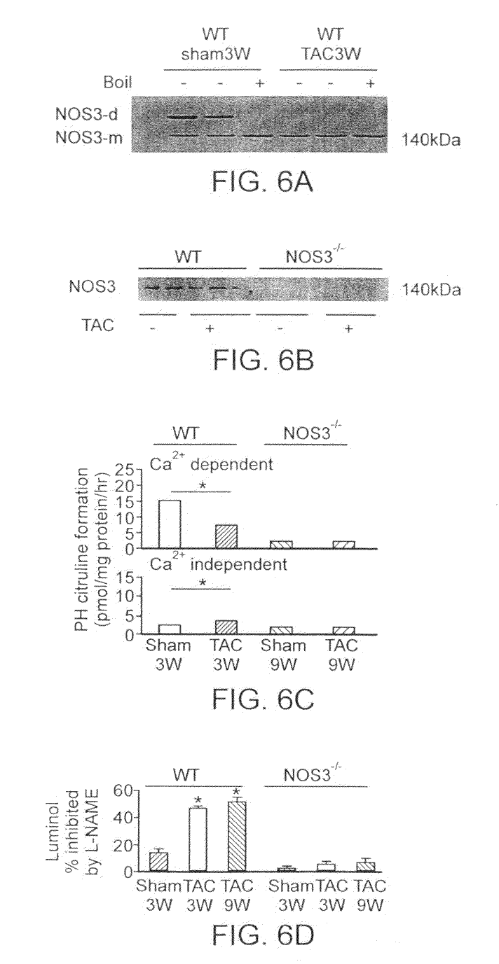

A potential explanation for the disparity between wild-type and NOS3^{-/-} responses to TAC was uncoupling of NOS3 in the wild-type hearts that could underlie enhanced reactive oxygen species generation. To test this, NOS3 was immuno-precipitated, and run in non-denaturing gels to assess dimer (coupled) versus monomer (uncoupled) forms⁷. Both monomer and dimer bands were present in wild-type sham-controls, whereas the monomer primarily existed in TAC hearts (FIG. 6A). Negative controls included boiled immuno-precipitates from wild-type myocardium to denature to the monomer form. TAC did not alter total NOS3 protein level as assessed by Western Blot (FIG. 6B). Further evidence for NOS uncoupling was obtained by a decline in Ca²⁺ dependent NOS activity (NOS3 and NOS1) in wild-type-TAC hearts (FIG. 6C), while Ca²⁺ independent activity (NOS2) was not altered. NOS3^{-/-} hearts displayed markedly reduced Ca²⁺-dependent but similar Ca²⁺-independent NOS activity.

Given the decline in NO-synthetic activity, it was next tested whether NOS-derived O₂⁻ increased with TAC. Tissue extracts were subjected to luminol chemiluminescence assay with or without co-incubation with the NOS inhibitor L-NAME (1 mM). The relative contribution of NOS was minimal under basal conditions, but rose markedly (˜50%) after 3- and 9-weeks TAC (FIG. 6D). Control data in NOS3^{-/-} hearts confirmed this to be from NOS3 and not the other NOS isoforms.

NOS uncoupling can occur due to a decline in BH4—a cofactor required for NOS to generate NO. BH4 levels in wild-type myocardium in shams versus 3-week TAC were therefore assessed by HPLC. BH4 was 9.8±2.6 nmol/g wet weight in shams and 3.9±1.1 in TAC (n=4-5 each group, p=0.007). There was also evidence of reduced BH4 synthesis reflected by a decline in the GTP-cyclohydrolase-1 product neopterin (11.5±7.3 to 1.1±0.3 nmol/g wet weight, p=0.03).

### Example 7

### Prevention of Remodeling, Reactive Oxygen Species Generation, and LV Dysfunction by BH4 but not H₄N

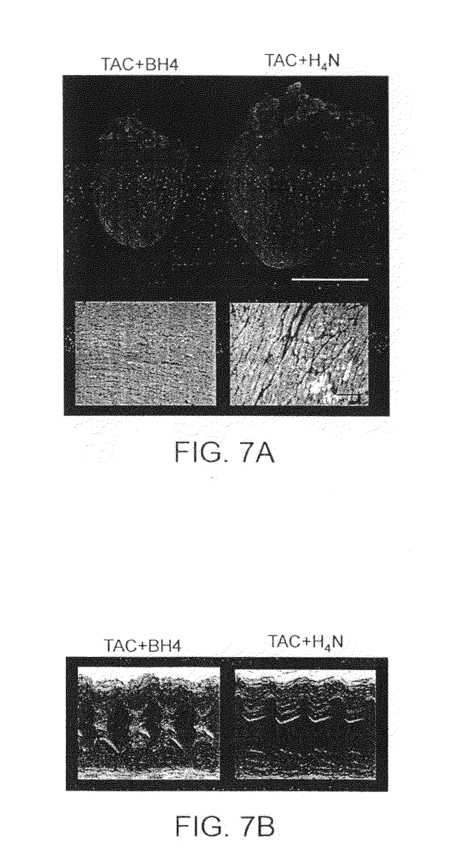

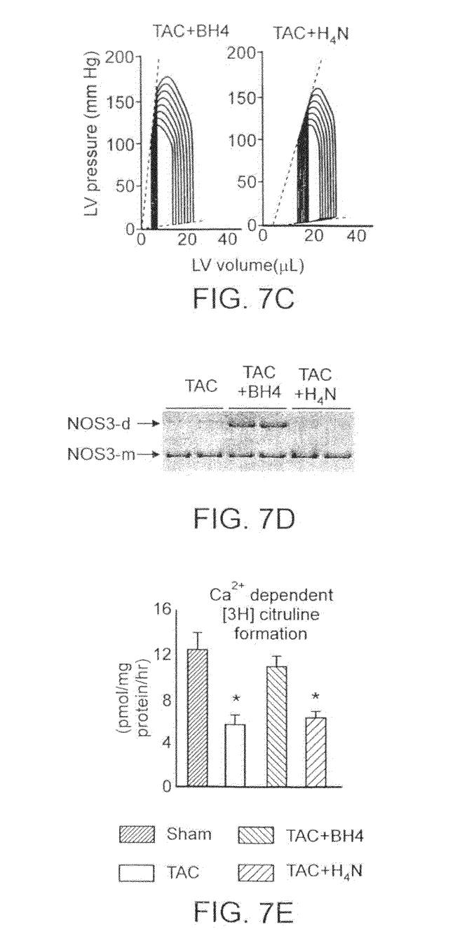

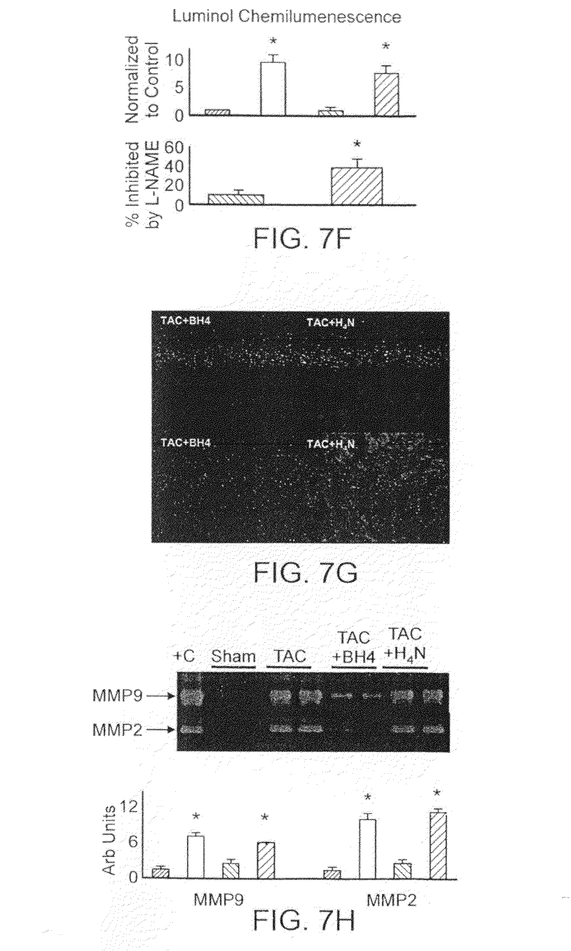

To further test the role of NOS3 uncoupling and reactive oxygen species generation in the wild-type-TAC (3-week) response, studies were performed in animals co-treated with oral BH4. As a control, parallel studies were conducted using tetrahydroneopterin (H₄N), which has similar anti-oxidant properties to BH4, but is not directly linked to NOS coupling and activity^{8,9}. BH4 but not H₄N treatment resulted in significantly blunted and morphologically concentric hypertrophic response to TAC with reduction in interstitial fibrosis (FIGS. 7A and B). In vivo left ventricular systolic and diastolic function in 3-week TAC mice treated with BH4 was improved compared to untreated controls despite an identical afterload increase. In contrast, H₄N treatment had no effect on functional or remodeling response to TAC (FIG. 7C—example PV loops and relations; Table 2 summary data).

TABLE 2
Effect of BH4 or H₄N supplementation on cardiac morphologic
and function response to TAC in non-transgenic (WT) mice.

| | WT TAC3W + BH4 | WT TAC3W + H₄N |
|---|---|---|
| BW and HW | n = 14 | n = 12 |
| Body Weight (BW) (g) | 26.6 ± 0.4 | 26.4 ± 0.6 |
| Heart Weight (HW) (mg) | 170.3 ± 3.9* | 231.3 ± 10.7 |
| Echocardiographic Analysis | n = 11 | n = 9 |
| Wall thickness (mm) | 1.09 ± 0.03 | 0.98 ± 0.03* |
| LV diameter Diastole (mm) | 2.87 ± 0.06* | 3.94 ± 0.19 |
| LV diameter Systole (mm) | 1.12 ± 0.06* | 2.70 ± 0.31 |
| Fractional Shortening (%) | 61.7 ± 1.7* | 33.8 ± 5.3 |
| Hemodynamics - PV loop | n = 5 | n = 4 |
| analysis | | |
| Heart RateR (min⁻¹) | 603.3 ± 22.9 | 501.2 ± 28.5 |
| LV Systolic Pressure (mmHg) | 193.0 ± 1.7* | 177.8 ± 4.6 |
| Arterial Elastance (mmHg/μL) | 10.6 ± 0.7 | 11.4 ± 0.7 |
| End-systolic Volume (μL) | 4.8 ± 1.7* | 24.0 ± 7.6 |
| End-diastolic Volume (μL) | 20.5 ± 1.9* | 38.8 ± 8.0 |
| Ejection Fraction (%) | 78.4 ± 5.8* | 43.4 ± 9.8 |
| dPdtmx (mmHg/s) | 17003 ± 1125* | 11154 ± 520 |
| Peak Power Index (mmHg/s) | 75.5 ± 10.5* | 31.8 ± 5.8 |
| End-systolic Elastance | 115.1 ± 9.6* | 41.0 ± 9.3 |
| (mmHg/μL · g) | | |
| dPdtₘₙ (mmHg/s) | −15743 ± 1325* | −10725 ± 971.0 |
| Tau (msec) | 3.5 ± 0.2* | 5.5 ± 0.36 |

Abbreviations are as in Table 1.
*P < 0.05 vs TAC3W + vehicle treatment.
Thus, from morphologic-functional standpoints, wild-type-TAC mice treated with BH4 (but not H₄N) displayed compensated concentric hypertrophy, phenotypes similar to NOS3^{-/-}-TAC animals.

Whether BH4 treatment restored NOS3 dimerization (FIG. 7D) and Ca²⁺-dependent activity (FIG. 7E) was next tested. Both were restored by BH4 but unaltered by H₄N co-treatment during TAC. In conjunction with these changes, reactive oxygen species generation markedly declined in BH4-treated animals as reflected by luminol chemiluminescence, DHE, and DCF assays (FIGS. 7F, 7G). Nitrotyrosine measured by ELISA fell to 8.48±0.98 μmol/mg protein, similar to wild-type-controls (c.f. FIG. 4D). BH4 treatment lowered the percent of luminol chemiluminescence inhibited by L-NAME to control levels. None of these changes were observed in mice treated with H₄N. Lastly, gelatin zymography revealed a marked reduction in gel lysis in BH4-treated TAC, but not in H₄N-treated TAC hearts (FIG. 7H).

As reported herein, NOS3 is a prominent source of myocardial reactive oxygen species induced by pressure-overload, and it is likely that NOS3 signaling is involved in the development of cardiac dilation, structural remodeling, and molecular and functional abnormalities. Under normal conditions, NOS3 generates nitric oxide which can have anti-hypertrophic influences. Pressure-load results in NOS3 uncoupling associated with reduced BH4 levels, transforming NOS3 activity to favor reactive oxygen species generation. Chronic BH4 administration restores NOS3 coupling, suppresses reactive oxygen species generation, and prevents the hypertrophy and remodeling changes induced by pressure-overload. These data identify NOS3 as a somewhat unexpected yet critical reactive oxygen species source in pressure-loaded hearts, and indicate a novel clinically applicable therapy that may prevent pathologic remodeling.

The lack of NOS3 not only blunted the hypertrophic response, but importantly prevented chamber dilation/remodeling. This disparity was present by 3-weeks and was more prominent after 9-weeks of TAC, with NOS3^{-/-} TAC hearts displaying concentric hypertrophy and enhanced systolic and diastolic function as compared with wild-type-TAC hearts. While chamber volumes declined in NOS3^{-/-} hearts, this did not reflect restrictive disease since diastolic pressures were unaltered, early filling rates were preserved, and importantly, end-systolic volumes were smaller. Rather, lack of NOS3 resulted in more compensated hypertrophy, with improved systolic and diastolic function. These results indicate that greater reactive oxygen species generation¹⁰ and associated activation of secondary signaling (e.g. MMPs, Akt) in wild-type-TAC hearts likely triggered chamber remodeling and decompensation.

Several lines of evidence supported NOS uncoupling due to TAC: the loss of NOS dimerization, a decline in BH4, a reduction in NOS NO-generating activity, and the increase in NOS-dependent reactive oxygen species generation. Restoration of these changes by BH4 treatment further support the mechanism, particularly as similar effects were not achieved with H₄N which has similar antioxidant properties to BH4. The demonstration that NOS3-uncoupling induced reactive oxygen species generation in cardiac hypertrophic pathophysiology provides for therapeutic interventions using BH4 or agents that enhance BH4 function. This approach is useful for the treatment of chronic hypertension, chamber dilation, fibrosis, and the development of functional depression.

Examples 1-7 show that BH4 is useful as a prophylactic for the prevention of cardiac chamber remodeling, muscle cell remodeling (e.g., myocyte hypertrophy), and molecular remodeling (e.g., re-expression of fetal genes).

### Example 8

### BH4 Reversed TAC-Induced Cardiac Hypertrophy

Studies reported in Examples 8-23, show that BH4 is not only useful as a prophylactic, but that it is also useful as a therapeutic for the treatment of cardiac indications. In vivo studies described below, show that BH4 reversed cardiac chamber remodeling, muscle cell remodeling (e.g., myocyte hypertrophy), and molecular remodeling (e.g., re-expression of fetal genes). These results indicate that BH4 is useful for the treatment of a variety of cardiac indications characterized by cardiac chamber remodeling, muscle cell remodeling (e.g., myocyte hypertrophy), and molecular remodeling, including cardiac hypertrophy and cardiac dilation.

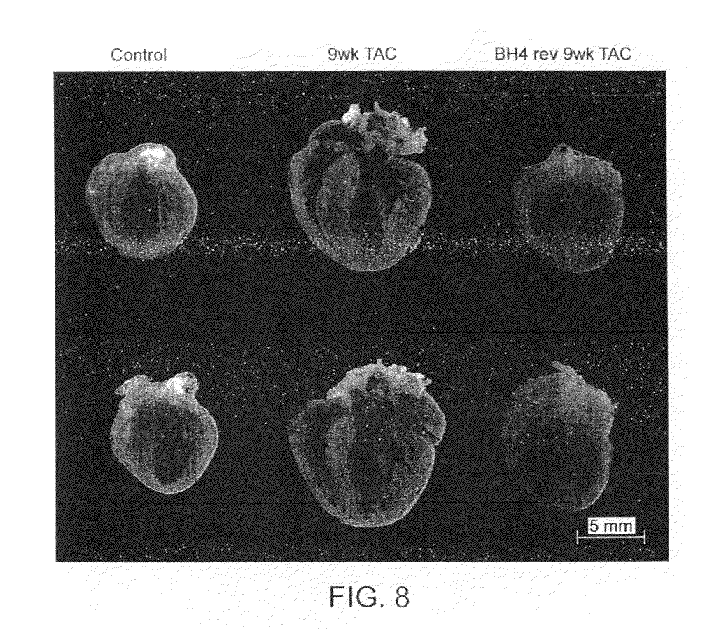

FIG. 8 shows that TAC induced dramatic cardiac hypertrophy within 9 weeks. Treatment with BH4 reversed cardiac hypertrophy in mice subjected to chronic trans-aortic constriction.

### Example 9

### BH4 Treatment Reversed TAC-Induced Posterior Wall Thickening

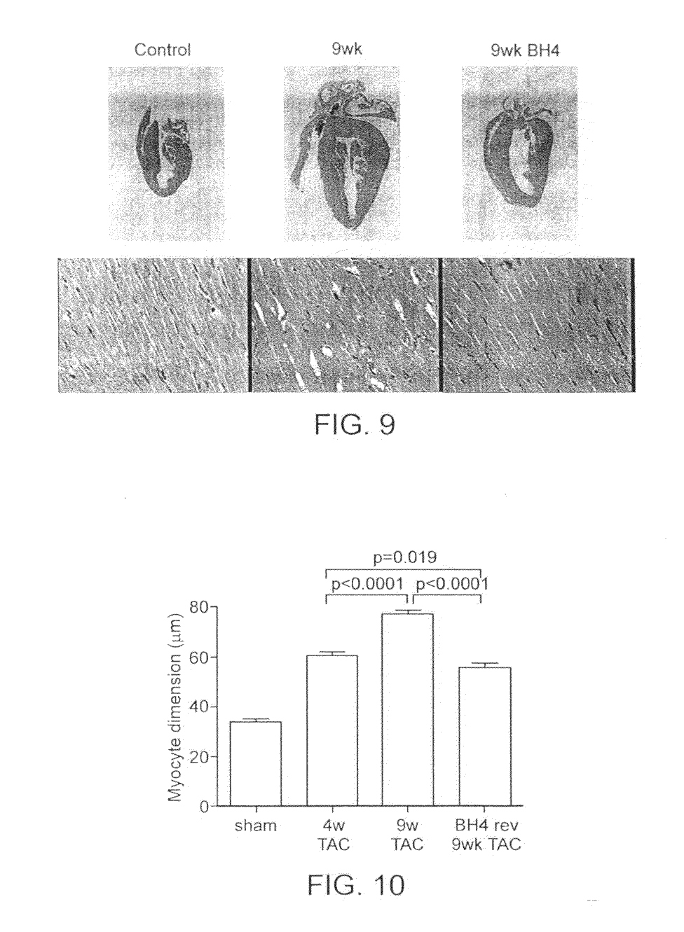

The effects of BH4 on cardiac fibrosis in wild-type mice subjected to chronic trans-aortic constriction is shown in FIG. 9. These results indicate that hearts under sustained pressure load show marked reversal of hypertrophy and reduced chamber size resulting from BH4 treatment relative to an untreated control. Myocyte size is markedly reduced (lower panels) compared to the untreated heart.

### Example 10

### BH4 Reverses Myocyte Hypertrophy

Cardiac myocyte hypertrophy increased steadily over the course of nine weeks during progressive TAC in untreated wild-type mice. In contrast, mice that received BH4 beginning at 4 weeks post-surgery showed a reduction in myocyte size at 9 weeks (FIG. 10). Myocyte cross sectional diameter was measured weekly following TAC. The myocyte size of BH4-treated mice at 9 weeks was similar to the size observed 3-weeks post-surgery in untreated animals. Thus, the reversal of left ventricle chamber hypertrophy is accompanied by a reversal of myocyte hypertrophy. Data was averaged over multiple cells imaged from 3-6 hearts in each condition.

### Example 11

### Cardiac Function is Enhanced by BH4 Treatment

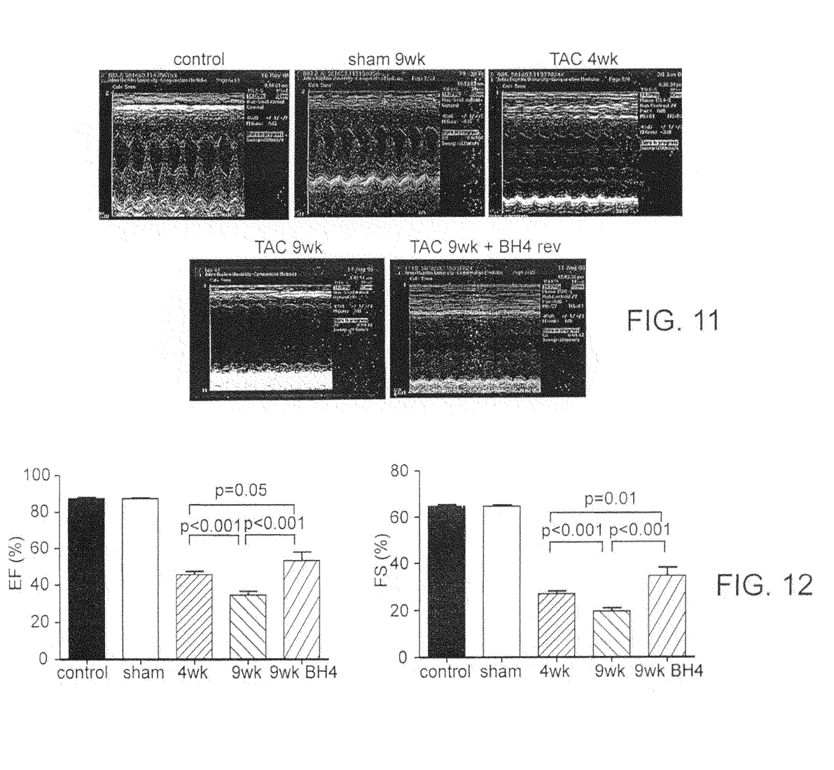

Over 4-9 weeks of progressive TAC, cardiac function as measured by echocardiography was significantly reduced (FIG. 11). Reductions in left ventricular systolic function, as measured by ejection fraction and fractional shortening, declined significantly in untreated animals (FIG. 12). Mice that received BH4 treatment had improved function compared with untreated hearts subjected to TAC. Ejection fraction and fractional shortening were both markedly enhanced in mice that received BH4 treatment beginning at week-4 and continuing through week-9 relative to untreated mice at 9-weeks post TAC surgery. This evidence indicates that BH4 treatment not only improved cardiac function, but actually reversed chamber dysfunction due to chronic pressure-overload (TAC).

### Example 12

### BH4 Treatment Reduced Cardiac Hypertrophy

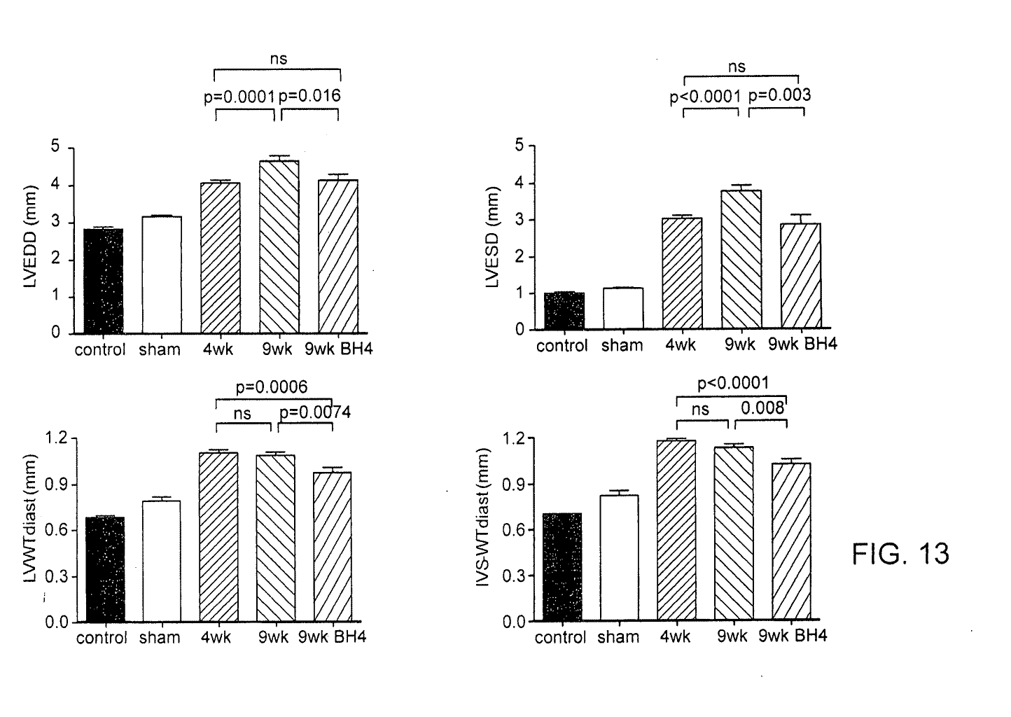

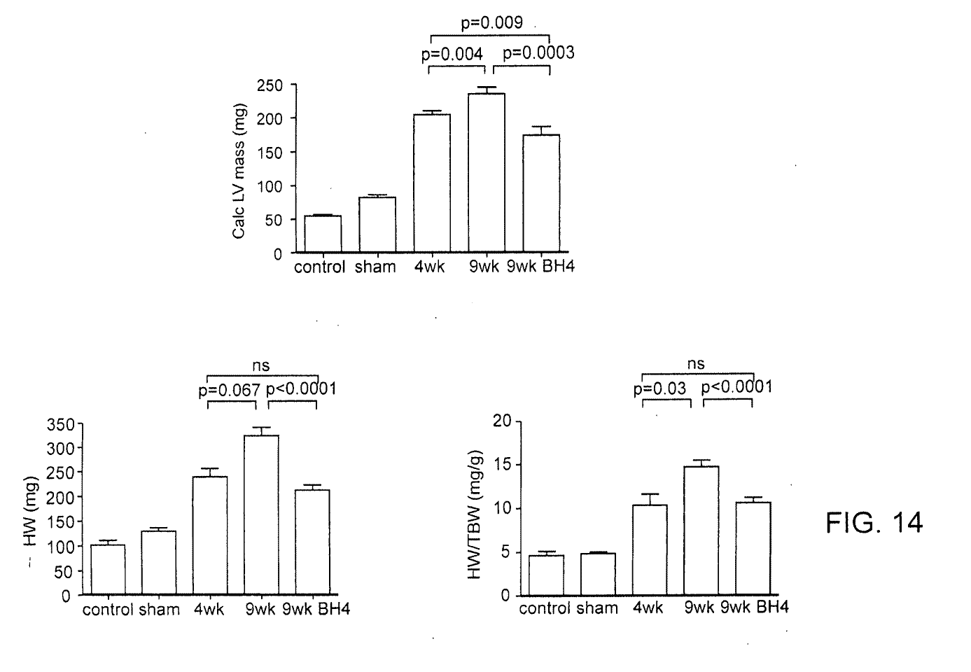

Echocardiography was used to measure wall thickness and other cardiac dimensions. BH4 treatment reduced diastolic wall thickening, left ventricular end-systolic and end-diastolic dimension relative to untreated TAC hearts at 9 weeks post-surgery (FIG. 13). Left ventricular mass, measured heart weight, and heart weight to body weight ratio was also reduced by BH4 treatment (FIG. 14).

### Example 13

### BH4 Treatment Reverses Nitric Oxide Synthase (NOS) Uncoupling

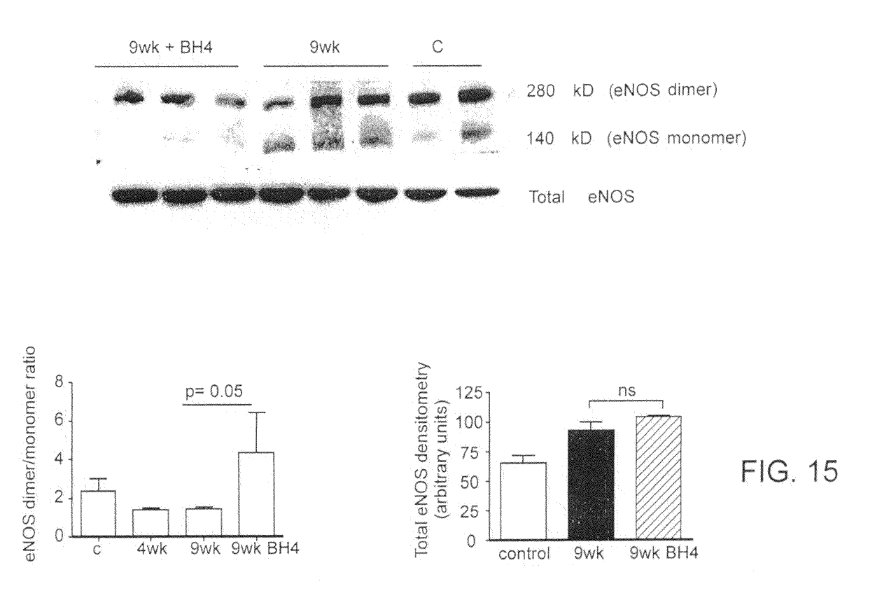

Nitric oxide synthase uncoupling is induced in advanced hypertrophic/dilated hearts at 4 weeks post-TAC. There was an increased level of the 140 kD monomer in hearts at 9 weeks post-TAC (FIG. 15). The increase in monomer was not seen at 9 weeks post Tac in mice that received BH4 treatment beginning at week four and continuing through week 9 (FIG. 15). These results indicated that NOS uncoupling was reversed by BH4 treatment. The ratio of dimer/monomer for eNOS increased with BH4 treatment although the total protein levels were unchanged.

### Example 14

### BH4 Treatment Reverses Myocardial Fibrosis

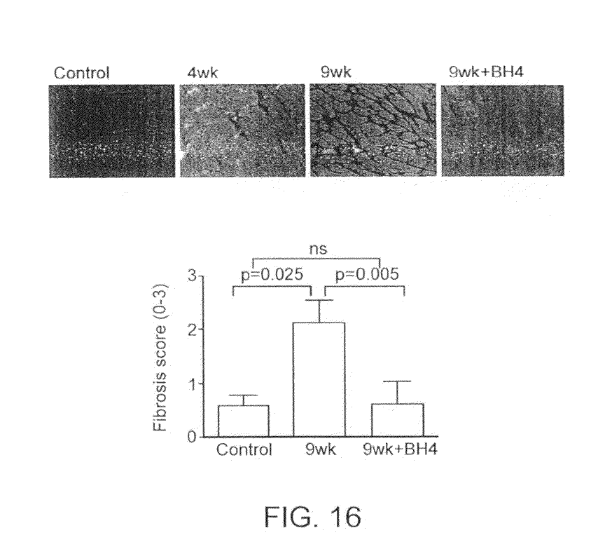

Myocardial histology showed minimal fibrosis present in normal control hearts. Interstitial fibrosis increased at 4 and 9 weeks following TAC (FIG. 16, upper panel). This interstitial fibrosis was reduced in mice that received BH4 treatment initiated at 4 weeks and continued for 5 weeks. In fact, fibrosis was reduced to virtually normal levels by BH4 treatment (FIG. 16, lower panel).

### Example 15

### Oxidative Stress was Reduced by BH4 Treatment

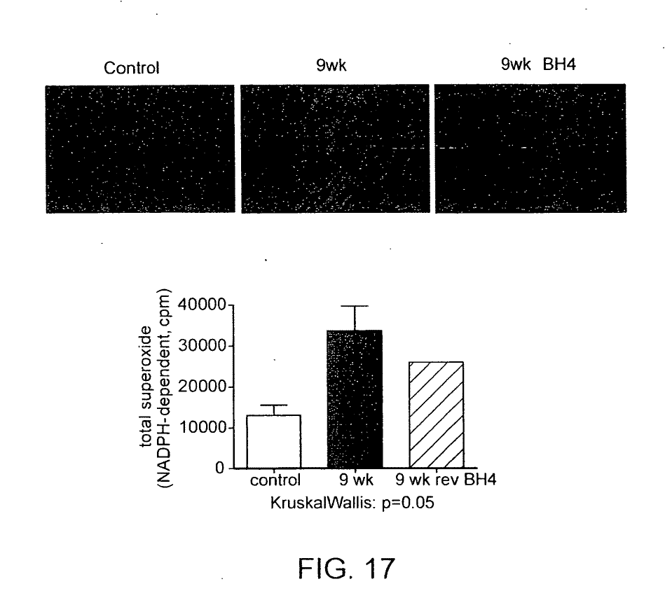

Superoxide levels were assayed by dihydroethidium (DHE) staining at 9 weeks post-TAC surgery in control animals and in animals treated with BH4. Superoxide levels are a marker of oxidative stress. Oxidative stress was markedly increased (light gray nuclei reflect positive DHE staining for superoxide) in hearts 9-weeks post-TAC. This increase in oxidative stress was largely reversed when BH4 treatment was initiated at week 4 and continued for 5 weeks (FIG. 17).

### Example 16

### BH4 Treatment Improves Cardiomyocyte Function

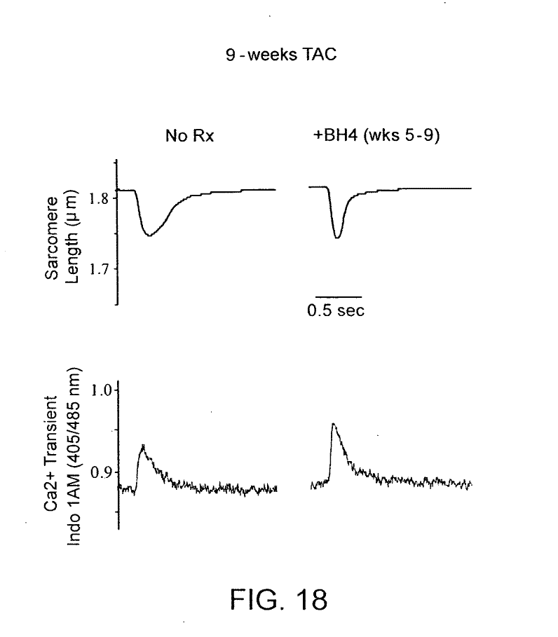

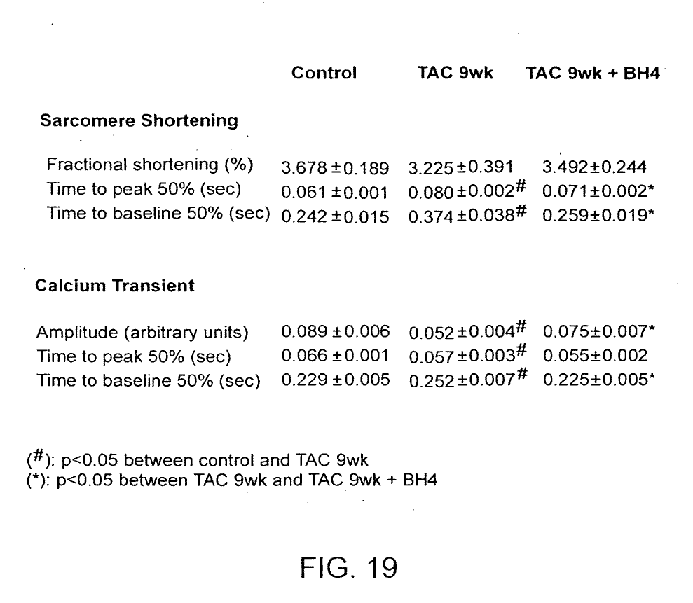

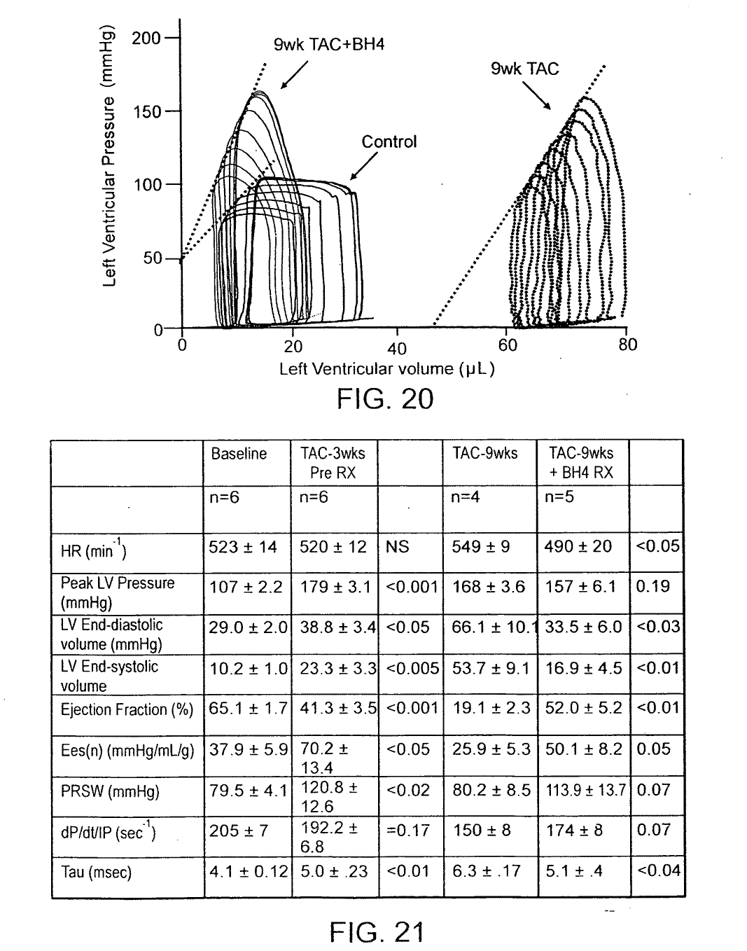

Chronic pressure overload reduced the kinetics of myocyte contraction and calcium handling as shown in FIG. 18. The kinetics of myocyte contraction and calcium handling were improved by BH4 treatment that was initiated at 4 weeks post-TAC and continued for 5 weeks as measured by myocyte shortening and calcium transient data (FIG. 19). The untreated chronic TAC heart displayed a marked increase in volume and depressed heart function (FIG. 20). In contrast, hearts treated with BH4 showed essentially normal heart volumes, and improved systolic function (FIG. 20). The degree of increased systolic pressure was similar in both treated and untreated 9 wk TAC hearts. FIG. 21 summarizes in vivo hemodynamic data in control animals, animals at 9 weeks TAC (n=4), and in BH4 treated animals at 9 weeks post-TAC (n=5).

### Example 17

### BH4 Reversed Oxidative Stress in the Myocardium

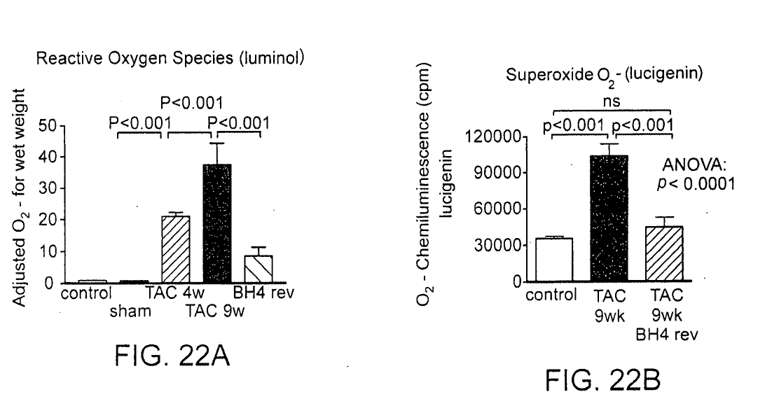

Consistent with results reported above for DHE staining, oxidative stress was also increased at 9-weeks post-TAC when superoxide levels were measured using a luminol and lucigenin assay (FIG. 22). Luminol is sensitive to oxidative stress including superoxide but also hydrogen peroxide and hydroxyl radical. Lucigenin is more specific to superoxide. Oxidant levels were measured in control, sham operated, and control and BH4 treated TAC animals at 4 weeks or 9 weeks post-surgery. TAC animals displayed a progressive increase in superoxide levels (oxidative stress). In contrast, the myocardial oxidative stress was reduced in TAC animals that received BH4 treatment beginning at 4 weeks post-TAC and continuing until 9 weeks post-TAC (FIG. 22). These data were confirmed by lucigenin assay, focusing on superoxide generation itself.

### Example 18

### BH4 Treatment Improved Nitric Oxide Synthase Activity and Reduced Superoxide Generation by NOS During Pressure Overload —Re-coupling NOS

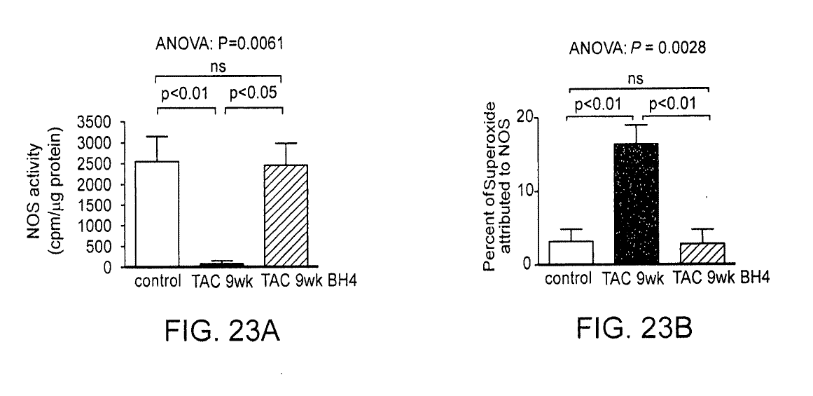

Calcium dependent nitric oxide synthase activity (combined activity of NOS3 and NOS1) was markedly reduced by 9 week TAC-coupled hypertrophy (FIG. 23A). Delayed BH4 treatment (weeks 5-9) improved the activity of nitric oxide synthase in myocardium exposed to sustained pressure overload. By “delayed BH4 treatment” is meant treatment that begins after symptoms of pathophysiology (e.g., NOS uncoupling, reactive oxygen species generation, myocyte dysfunction, chamber remodelling) are present. While NOS activity declined with TAC, its generation of superoxide increased (FIG. 23B). This was significantly reduced by delayed BH4 treatment. Together these data support reversal of NOS uncoupling—or NOS recoupling—produced by BH4 treatment in hearts with preexisting advanced hypertrophy/remodeling.

### Example 19

### BH4 Treatment Enhances Cardiac Function and Reduces Cardiac Hypertrophy

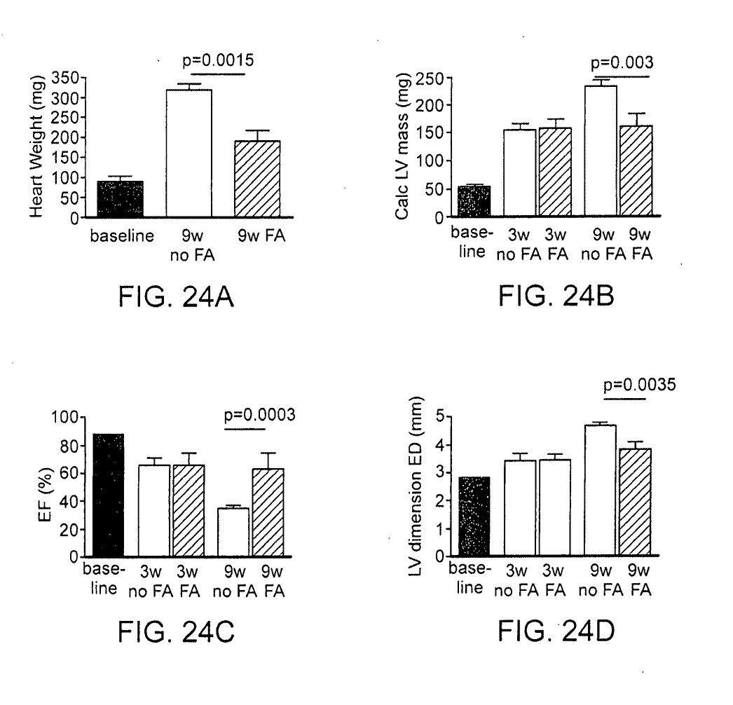

Folate can enhance intrinsic BH4 levels by enhancing the salvage pathway. The salvage pathway converts oxidized BH4 to the reduced form, enabling it to work properly as a NOS cofactor. Folate treatment prevented advanced cardiac hypertrophy, dilation, and improved cardiac function in mice exposed to 9 weeks of TAC. TAC animals were treated with folate (10 mg/kg/day) or with placebo for up to 9 weeks post-surgery. As shown in FIG. 24 ejection fraction significantly improved (p=0.05), LV mass was reduced, and cardiac dilation was diminished in animals receiving folate treatment (all p<0.05).

### Example 20

### Anti-Oxidants Restore the Ability of Hypertrophied Tissues to Respond to PDE5a Inhibition

As noted in examples 17-19, PDE5 inhibition both prevented and reversed cardiac hypertrophy and dysfunction induced by chronic pressure overload. This effect is markedly diminished in hearts in which NOS3 is either genetically absent (NOS3^{-/-}) or inhibited (e.g. L-NAME). This indicates that the generation of NO and subsequent activation of soluble guanylase cyclase to generate cGMP is needed for normal regulation of cardiac stress response by PDE5. By restoring NOS3 gene expression in hearts in which this was genetically absent, the ability of PDE5-inhibition to blunt acute catecholamine stress is restored. The ability of BH4, anti-oxidants, or soluble guanylase cyclase activators to enhance NOS function or cGMP production improves the efficacy of PDE5 inhibitors in cardiac conditions.

Adult myocytes isolated from wild-type C57/B16 mouse hearts responded to β-adrenergic stimulation by isoproterenol by increased contraction measured as sarcomere shortening. Isolated wild-type (C57B16) myocytes were exposed to isoproterenol (10 micromolar) and increased sarcomere shortening was observed by real-time fast Fourier transform (FFT) analysis. The addition of a PDE5a inhibitor (sildenafil, 0.1) blunts the increased shortening by ˜25%.

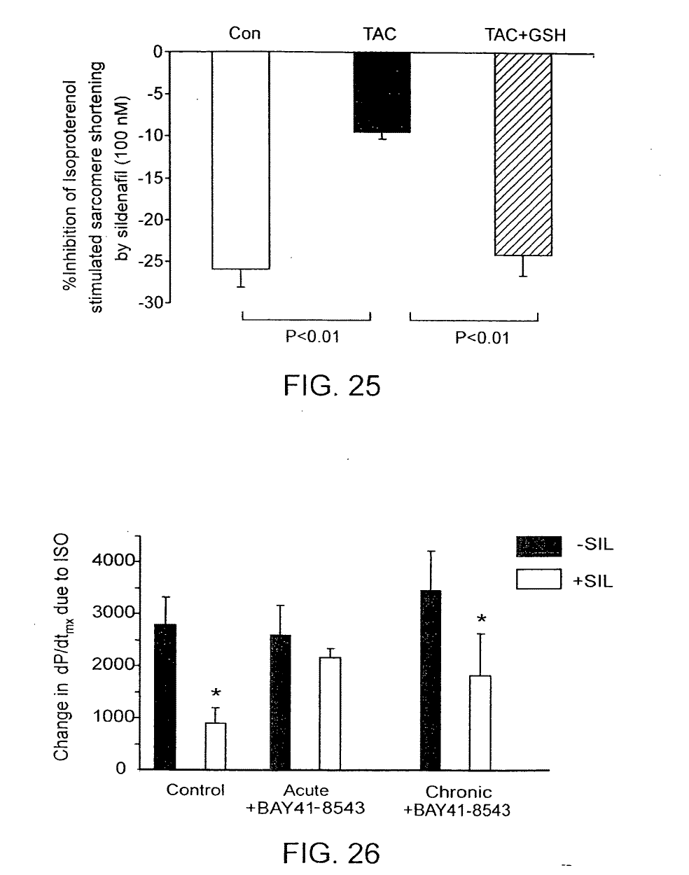

In myocytes isolated from chronically hypertrophied and dilated hearts (3-4 weeks f TAC), the ability of sildenafil to blunt the isoproterenol response was markedly diminished (p=0.04). If these cells were first treated with the anti-oxidant (reduced glutathione), then the capacity of PDE5a inhibition to blunt the beta-adrenergic response was recovered (FIG. 25). It is likely that PDE5a inhibition effects are modulated by redox, and that agents that alter redox enhance nitric oxide signaling. BH4, antioxidants, and folate will likely restore the ability of chronically hypertrophied and dilated hearts to respond to PDE5a inhibition.

### Example 21

### NOS-Related Signaling Enhancers Improve Modulation of Acute Adrenergic Stress by PDE5a Inhibitors in Hearts with NOS Inhibition

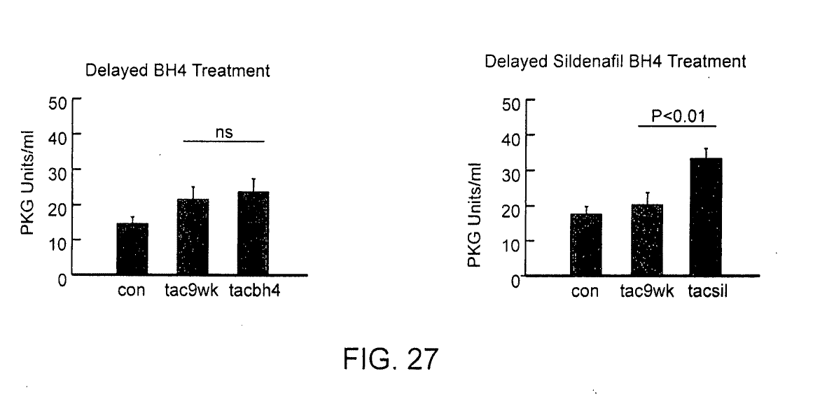

Regulation of acute beta-adrenergic stress by PDE5a inhibition (resulting in a rise in cGMP and activation of protein kinase G) requires the activity of nitric oxide synthase (NOS). In hearts lacking the NOS3 isoform or where NOS is inhibited (i.e. by L-NAME), PDE5a inhibition does not suppress an acute β-adrenergic stimulant such as isoproterenol (ISO). This can be offset by chronic distal activation of the NO target protein soluble guanylate cyclase 9sGC). This is shown in FIG. 26. At the left are bars depicting the contractility response of an intact heart to ISO before and after co-administration of SIL. The ISO response is normally blunted by nearly 80%. The middle bars show data from mice with NOS inhibited by L-NAME (1 mg/L in drinking water). The ISO response is no longer inhibited by SIL. As reported herein, even if soluble guanylate cyclase is directly stimulated (which is the protein responsible for generating cGMP following NO stimulation), SIL still does not counter the ISO response. However, if one continues to treat hearts with the soluble guanylate cyclase activator (BAY 41-8543) for a week—all the while continuing NOS inhibition with L-NAME, the ability of SIL to suppress adrenergic stimulation is restored. These results indicate that enhancers of NOS-related signaling can increase the physiologic regulation of heart function by PDE5a inhibitors.

### Example 22

### BH4 and Sildenafil Act Through Different Mechanisms

Chronic BH4 treatment and chronic PDE5a (sildenafil) inhibitor treatment act through different mechanisms in hearts exposed to sustained pressure overload as shown in FIG. 27. Protein kinase G activity increased nominally over time in 9-week TAC hearts, although this falls just short of statistical significance. However, in hearts receiving sildenafil during weeks 5-9, PKG activity significantly increased (right set of bars). Sildenafil likely increased PKG activity by inhibiting cGMP hydrolysis, increasing levels of cGMP and thereby increasing levels of its downstream target kinase PKG. In contrast, BH4 reversed hypertrophic remodeling without enhancing PKG activity. Rather, the prior data supports an effect on reducing oxidant stress and re-coupling nitric oxide synthase. Given that the two compounds work through different mechanisms, administration of a combination of BH4 and sildenafil, or another PDE5 inhibitor, should have a synergistic effect on cardiac hypertrophy.

These experiments were carried out using the following materials and methods. Such methods and their results are related to those described in Takimoto et al., Circ Res 2005; 96:100-109; and Takimoto et al, Nature Medicine 2005; 11(2):214-22, each of which is expressly incorporated by reference in its entirety.

### Animals and Preparation.

Male NOS3 null mice (NOS3^{-/-}) and C57/BL6 WT controls (8-11 weeks, Jackson Labs, Bar Harbor, Me., USA) were used. Pressure overload was produced by transverse aorta constriction (TAC). Briefly, The aortic arch was isolated by entering the extrapleural space between the second and third rib, and afterwards the transverse aorta was isolated between the right and left carotid arteries. A 7-0 prolene suture ligature was tied around the transverse aorta against a 27-gauge needle to produce a 65-70% constriction after the removal of the needle. After closing the chest, animals were extubated, and a subcutaneous injection of morphine sulphate 0.2 mg, was administered before they returned to their cages. Animal's ventilatory and circulatory status was checked every 30 minutes during the first 2 hours. Sham-operated mice underwent the same operation except for aortic constriction. All TAC-animals demonstrated after 4 weeks profound cardiac hypertrophy, and non-decompensated dilation. Animals were randomized to receive placebo or BH4 (120 mg/kg/d) for the next 5 weeks. At the end of the study, i.e. at 9 weeks, animal were sacrificed to receive tissue or underwent in vivo PV-loop analysis. TAC acutely increased LV systolic pressure by 67.2±0.3 mmHg in WT, and 69.0±1.5 mmHg in NOS3^{-/-} mice (p=ns between groups). Control mice were subject to sham operations, and animals were studied 3-9 weeks following surgery. An additional group of wild-type animals were subjected to TAC for 3 weeks while co-treated with oral tetrahydrobiopterin (BH4) (Sigma-Aldrich) (1 mg/g food) mixed in their rodent chow, providing 5 mg/day based on 4-6 g daily diet⁸. Control studies were also performed using oral tetrahydroneopterin (H₄N) (Schircks Laboratories) (1 mg/g food)⁸, an antioxidant that does not directly participate in NOS3 coupling⁹.

Two different BH4 treatment protocols are used. For experiments related to FIGS. 1-7, where prevention of cardiac indications is described BH4 is provided orally as a treatment starting 2 days after TAC surgery (once mice are again eating oral diet). For those figures related to treatment of established cardiac indications, FIGS. 8-23, BH4 is provided orally but this is delayed for 4 weeks to establish substantial cardiac hypertrophy, remodeling, and NOS-uncoupling. Then the treatment is provided for the remaining 5 week period.

For folate studies, C57/BL6 WT were treated with folate (10 mg/kg/day) or with placebo for a 9 week period during which time the hearts were exposed to TAC.

### Echocardiography.

In vivo cardiac morphology was assessed by transthoracic echocardiography (Acuson Sequoia C256, 13 MHz transducer, Siemens) in conscious mice. M-mode left ventricular (LV) end-systolic and end-diastolic dimensions were averaged from 3-5 beats. Left ventricular ejection fraction (LVEF) and percent fractional shortening (% FS) were calculated as: LVEF=[(LVEDD)³-(LVESD)³]/(LVEDD)³×100; % FS=(LVEDD−LVESD)/LVEDD×100. Wall thickness of lateral freewall and intraventricular septum were averaged. Studies and analysis were performed blinded to heart condition.

### In Vivo Hemodynamics

In vivo left ventricular (LV) function was assessed by pressure-volume catheter^{12,13}. Mice were anesthetized with 1-2% isoflurane, urethane (750-100 mg/kg, i.p.), etomidate (5-10 mg/kg, i.p.), and morphine (1-2 mg/kg, i.p.), underwent tracheostomy, and were ventilated with 6-7 μL/g tidal volume and 130 breaths/min. Volume expansion (12.5% human albumin, 50-100 μL over 5 min) was provided through a 30 G cannula via the right external jugular vein. The left ventricular apex was exposed through an incision between the 7-8^{th} rib, and a catheter, specifically a 1.4 Fr PV catheter (SPR 839, Millar Instruments, Inc.), was advanced through the apex to lie along the longitudinal axis. Absolute volume was calibrated, and pressure-volume data was measured at steady state and during transient reduction of venous return as reported¹³

### Fetal Gene Expression Dot Blot Analysis

Gene expression for A and B-type natriuretic peptides, β-myosin heavy chain, α-skeletal actin, and the calcium handling proteins—phospholamban (PLB) and SR—Ca²⁺-ATPase (SERCA2a) were performed by dot-blot analysis as described¹⁴.

### Reactive Oxygen Species and Nitrotyrosine Analysis

Reactive oxygen species generation was examined by several independent methods. Superoxide production in left ventricular tissue homogenates was determined by luminol-enhanced chemiluminescence (EMD Biosciences). Flash frozen myocardium was homogenized in iced PBS buffer, centrifuged, and the precipitate re-suspended in assay buffer to a final concentration of 100 μM luminol following manufacturer's instructions. Phorbol-12-myristate-13-acetate or other oxidase stimulators were not used in the assay. Data were normalized by sample weight. In addition, fresh frozen left ventricular myocardium (8 μm slices) was incubated for 1 hour at 37° C. with florescent dyes 2′,7′-dichlorodihydro-fluorescein diacetate (DCF; Molecular Probes; 4 μM) (Invitrogen Corp.) reflecting hydrogen peroxide formation (DCF staining localizes principally to mitochondria within myocytes), dihydroethidium (DHE; Molecular Probes; 2 μM) which assesses O₂⁻ formation (typically nuclear localization), and nitrotyrosine formation (polyclonal nitrotyrosine Ab, 1:100; Upstate), which can reflect formation of ONOO⁻. Imaging was performed on a Zeiss inverted epifluorescence microscope attached to an argon-krypton laser confocal scanning microscope (UltraVIEW, Perkin Elmer Life Sciences, Inc.). The excitation/emission spectrum for DHE was 488 and 610 nm, respectively, with detection at 585-nm, and for DCF was 480 and 535 μm, respectively, with detection at 505-nm. Nitrotyrosine was also quantitatively assessed by ELISA assay (Oxis International).

### HPLC Analysis of Oxidative Stress, BH4, and Energy Metabolites

Mice hearts were quickly excised, immersed in liquid nitrogen and then subjected to the organic solvent deproteinization procedure as described¹⁵. Aliquots of each deproteinized tissue extract (10% weight/volume) were filtered through a 0.45 μm HV-Millipore filter and then assayed by ion-pairing HPLC as reported¹⁵. Ultrapure HPLC standards were provided by Sigma. For (6R)-5,6,7,8-tetrahydrobiopterin (BH4) and D-(+)-neopterin, separation was carried out on 20 μl using a 5 μm particle size column, the Kromasil 250×4.6 mm, provided with its own guard column (Eka Chemicals AB) using a step gradient from buffer A (10 mM tetrabutylammonium hydroxide, 10 mM KH₂PO₄, 0.25% methanol, pH 7.00) to buffer B (2.8 mM tetrabutylammonium hydroxide, 100 mM KH₂PO4, 30% methanol, pH 5.50). The flow rate was 1.2 ml/min and column temperature was held constant at 18° C. Under these conditions, BH4 is eluted isocratically with k′ of 3.02 and neopterin with a k′ of 8.56, where k′=V−V₀/V₀ (V=elution volume of compound, V₀=void volume of chromatographic system). The lower assay limit was 0.5 μM, corresponding to 10 pmol/20 μl injected volume. Species identification was made by matching retention times and absorption spectra to freshly prepared ultra-pure standards, and if needed co-chromatograms performed by adding known standards to the biological samples. Concentration was calculated from the standard run data at wavelengths corresponding to peak absorption of each substance.

### Tissue Histology

Formalin fixed (10%) myocardium or fresh frozen myocardial specimens preserved in OCT were paraffin embedded and prepared for histologic analysis using hematoxalyn/eosin stain to assess myocyte size, inflammation, and other gross microscopic pathology, and PAS methenamine or Masson-trichrome for interstitial fibrosis. 5-8 μm slices were stained, and Photomicrographs quantified to assess mean cardiomyocyte diameter and interstitial collagen fraction using computer assisted image analysis (Adobe Photoshop 5.0; Adobe, NIH Image J). Average data reflect results from 4 hearts in each group, (>30 cells).

### Cardiac Gelatinase Analysis

In vitro gelatin lysis by MMP-2 and MMP-9 was assessed by zymography. Briefly, modified Laemmli buffer without mercaptoethanol was added to lysed tissue samples and loaded on a 10% gelatin (Invitrogen Corp.). After electrophoresis, gels were washed twice with renaturing buffer at room temperature followed by developing buffer (Invitrogen Corp.), then stained to visualize lytic bands (SimplyBlue, Invitrogen Corp.).

### Determination of NOS Dimerzation and Activity

SDS-resistant NOS3 dimers and monomers were assayed using low-temperature SDS PAGE under reducing or non-reducing conditions, as described previously⁷. NOS3 was immunoprecipitated as described¹⁶ and the resulting samples added to fivefold Laemmli buffer (0.32 mol/l Tris-HCl, pH 6.8, 0.5 mol/l glycine, 10% SDS, 50% glycerol, and 0.03% bromophenol blue) in non-reducing gel (no 2-mercaptoethanol) to identify dimer dissociation due to reduced disulfide bridges. To provide fully denatured control lanes, samples were boiled for 15 minutes prior to loading. Electrophoresis was performed using Tris glycine 6% gels (Invitrogen Corp.), and gels and buffers maintained in an ice bath at 4° C. and stained (SimplyBlue, Invitrogen Corp.). Calcium dependent and independent NOS activity was determined from myocardial homogenates by [3H] L-arginine to [3H] citrulline conversion (Sigma-Aldrich) as described¹⁶. An alternative approach involved more direct Western blot analysis without requiring initial NOS IP. Cold SDS-Page Western blot analysis was performed using a self-made 74% SDS-Tris gel. After overnight running on ice, the gel was transferred for 3 h to nitrocellulose membrane. The primary eNOS antibody was used in a 1:350 solution (Santa Cruz, Ca). Bound antibodies were detected with horseradish peroxidase-conjugated anti-mouse IgG and visualize with an enhanced chemiluminescence detection system.

### Akt Activation.

Akt activation was assessed by Western blotting for total Akt, and S379 phosphorylated Akt (1:1000 dilution) (Cell Signaling Technology). Protein concentration was determined by bicinchoninic acid method and primary antibodies visualized by horseradish peroxidase-conjugated secondary antibodies and enhanced chemiluminescence (Pierce Biotechnology).

### Statistical Analysis

All values were expressed as mean±SEM. Group data were compared using one-way or two-way ANOVA, (with genotype and ±TAC as categories), and a Tukey's post-hoc multiple comparisons test for between group differences. Unless specifically noted, analysis was performed with n=4-6 for each group in a given assay.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Iwanaga, et al., (2002) J. Am. Coll. Cardiol. 39:1384-1391.
2. Rajagopalan, et al., (1996) J. Clin. Invest 98:2572-2579.
3. Leslie, et al., (2003) EMBO J. 22:5501-5510.
4. Matsui, et al., (2002) J. Biol. Chem. 277:22896-22901.
5. Matsui, et al., (2003) Cell Cycle 2:220-223.
6. Matsui, et al, (2002) J. Biol. Chem. 277:22896-22901.
7. Zou, et al., (2002) J. Clin. Invest 109:817-826.
8. Landmesser, et al., (2003) J. Clin. Invest 111:1201-1209.
9. Heitzer, et al., (2000) Circ Res 86:E36-E41.
10. Ferdinandy, et al., (2000) Circ. Res. 87:241-247.
11. Takimoto, et al., (2002) FASEB J. 16:373-378.
12. Georgakopoulos, et al., (1998) Am. J. Physiol 274:H1416-H1422.
13. Isoda, et al., (2003) FASEB J 17:144-151.
14. Liao, et al., (2001) Proc. Natl. Acad. Sci. U.S.A. 98:12283-12288.
15. Lazzarino, et al., (2003) Anal Biochem 322:51-59.
16. Champion, et al., (2004) Circ. Res. 94:657-663.

1. A method of treating a cardiac disease or disorder in a subject, the method comprising administering to the subject a compound that modulates NOS activity, wherein the method treats a cardiac disease or disorder.

2. The method of claim 1, wherein the compound modulates NOS3 activity.

3. The method of claim 2, wherein the compound reduces NOS3-dependent generation of reactive oxygen species.

4. The method of claim 1, wherein the compound is tetrahydrobiopterin.

5. The method of claim 1, wherein the method increases NO production.

6. (canceled)

7. A method of treating a cardiac disease or disorder in a subject, the method comprising administering to the subject a compound comprising an effective amount of tetrahydrobiopterin, wherein the compound modulates NOS3 activity.

8. The method of claim 1, wherein the cardiac disease or disorder is selected from the group consisting of cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure.

9. The method of claim 7, wherein the compound reduces NOS3-dependent generation of reactive oxygen species.

10. The method of claim 1, wherein the method reduces or reverses cardiac chamber remodeling.

11. The method of claim 1, wherein the method reduces or reverses cardiac dilation.

12. The method of claim 1, wherein the method reduces or reverses cardiac muscle cell remodeling.

13. The method of claim 1, wherein the method reduces myocyte hypertrophy.

14. The method of claim 1, wherein the method reduces or reverses molecular remodeling.

15. The method of claim 1, wherein the method reduces or reverses myocardial fibrosis.

16. The method of claim 1, wherein the method reduces or reverses oxidative stress.

17. A method of enhancing cardiac function in a subject having a cardiac condition selected from the group consisting of cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure, the method comprising administering to the subject an effective amount of tetrahydrobiopterin, wherein the administration of a compound comprising an effective amount of an NOS modulator enhances cardiac function.

18. (canceled)

19. The method of claim 17, wherein the NOS modulator is tetrahydrobiopterin.

20. The method of claim 17, wherein the method reduces or reverses cardiac chamber remodeling.

21. The method of claim 17, wherein the method reduces or reverses cardiac dilation.

22. The method of claim 17, wherein the method reduces or reverses cardiac muscle cell remodeling.

23. The method of claim 17, wherein the method reduces myocyte hypertrophy.

24. The method of claim 17, wherein the method reduces or reverses molecular remodeling.

25. The method of claim 17, wherein the method reduces or reverses myocardial fibrosis.

26. The method of claim 17, wherein the method reduces or reverses oxidative stress.

27. The method of claim 22, wherein the method reduces re-expression of a fetal gene.

28-47. (canceled)

48. The method of claim 1 further comprising the step of administering to the subject a PDE5 inhibitor.

49. The method of claim 48, wherein the combination of BH4 and PDE5 have a synergistic therapeutic effect.

50-55. (canceled)

56. A method of enhancing cardiac function in a subject having a cardiac condition selected from the group consisting of cardiac hypertrophy, reduced systolic function, reduced diastolic function, maladaptive hypertrophy, heart failure with preserved systolic function, diastolic heart failure, hypertensive heart disease, aortic stenosis, hypertrophic cardiomyopathy, post ischemic cardiac remodeling and cardiac failure, the method comprising administering to the subject an effective amount of folic acid or a metabolite thereof, wherein the administration of the compound enhances cardiac function.

57. The method of claim 56, wherein the method further comprises administering to the subject sildenafil.

58. The method of claim 56, wherein the method further comprises administering to the subject BH4.

59. A method of treating or preventing a cardiac disease or disorder in a subject, the method comprising administering to the subject an effective amount of a combination of tetrahydrobiopterin and at least one compound selected from the group consisting of a PDE5 inhibitor, an anti-oxidant, folate, YC-1, BAY 58-2667, BAY 41-2272, or BAY-41-8543, wherein the administration of the combination treats or prevents a cardiac disease or disorder.

60-89. (canceled)

90. A pharmaceutical composition comprising:
(i) an effective amount of tetrahydrobiopterin in a pharmaceutically acceptable excipient, wherein the pharmaceutical pack is labeled for use in the treatment or prevention of a cardiac disease or disorder; or
(ii) an effective amount of tetrahydrobiopterin and at least one compound selected from the group consisting of at least one compound selected from the group consisting of a PDE5 inhibitor, an anti-oxidant, folate, YC-1, BAY 58-2667, BAY 41-2272, or BAY-41-8543, in a pharmaceutically acceptable excipient, wherein the pharmaceutical pack is labeled for use in the treatment or prevention of a condition selected from the group consisting of a cardiac disease or disorder; or
(iii) an effective amount of folic acid or a metabolite thereof in a pharmaceutically acceptable excipient, wherein the pharmaceutical pack is labeled for use in the treatment or prevention of a condition selected from the group consisting of a cardiac disease or disorder.

91-96. (canceled)

97. A method of preventing a cardiac disease or disorder in a subject, the method comprising administering to the subject a compound that modulates NOS activity, wherein the method treats a cardiac disease or disorder.

98-112. (canceled)

113. A kit for the treatment of a cardiac disease or disorder comprising:
(i) tetrahydrobiopterin and at least one of a PDE5 inhibitor, an anti-oxidant, folate, YC-1, BAY 58-2667, BAY 41-2272, or BAY-41-8543 and directions for their use in the treatment or prevention of a cardiac disease or disorder; or
(ii) folic acid or a metabolite thereof and at least one of a PDE5 inhibitor, an anti-oxidant, or tetrahydrobiopterin, and directions for their use in the treatment or prevention of a cardiac disease or disorder.

114. (canceled)
